(19) United States
(12) Patent Application Publication
(10) Pub. No.: US 2016/0083654 A1
(43) Pub. Date: Mar. 24, 2016

(54) LIQUID CRYSTAL COMPOSITION AND LIQUID CRYSTAL DISPLAY COMPRISING THE LIQUID CRYSTAL COMPOSITION

(71) Applicant: Samsung Display Co., Ltd., Yongin-City (KR)

(72) Inventors: Fusayuki Takeshita, Yongin-City (KR); Jaejin Lyu, Yongin-City (KR); Siheun Kim, Yongin-City (KR)

(21) Appl. No.: 14/630,892

(22) Filed: Feb. 25, 2015

(30) Foreign Application Priority Data
Sep. 22, 2014 (KR) 10-2014-0126050

(51) Int. Cl. C09K 19/30; C07C 22/00

(52) U.S. Cl. CPC C09K 19/3066; C09K 19/3003; C09K 19/3001; C07C 22/00; C09K 2019/3004; C09K 2019/3009; C09K 2019/301; C07C 2101/14

(57) ABSTRACT

A liquid crystal composition comprises at least one of first liquid crystal compounds represented by Formula 1, and a liquid crystal display including the liquid crystal composition:

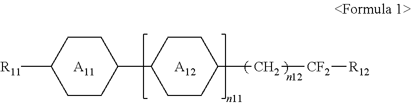

Wherein

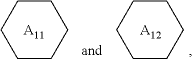

R₁₁ and R₁₂, and n11 and n12 are defined as in the specification.

### CLAIM OF PRIORITY

This application makes reference to, incorporates into this specification the entire contents of, and claims all benefits accruing under 35 U.S.C. §119 from an application LIQUID CRYSTAL COMPOSITION AND LIQUID CRYSTAL DISPLAY COMPRISING THE LIQUID CRYSTAL COMPOSITION earlier filed in the Korean Intellectual Property Office on Sep. 22, 2014 and there duly assigned Serial No. 10-2014-0126050.

### BACKGROUND OF THE INVENTION

1. Field of the Invention

The present invention relates to a liquid crystal composition and a liquid crystal display including the liquid crystal composition.

2. Description of the Related Art

Liquid crystal displays that are convenient to carry and have low power consumption are one of the most widely used flat panel displays.

A liquid crystal display includes two substrates with a pixel electrode or a common electrode disposed thereon, and a liquid crystal layer disposed between the two substrates. When a voltage is applied to the pixel electrode or the common electrode to generate an electric filed in the liquid crystal layer, the alignment of liquid crystal molecules in the liquid crystal layer is changed to alter the polarization of light passing through the liquid crystal layer, and consequently, an image is displayed on the liquid crystal display.

### SUMMARY OF THE INVENTION

The present invention relates to a liquid crystal composition and a liquid crystal display including the liquid crystal composition.

Additional aspects will be set forth in part in the description which follows and, in part, will be apparent from the description, or may be learned by practice of the presented embodiments.

According to one or more embodiments of the present invention, a liquid crystal composition may include at least one of first liquid crystal compounds represented by Formula 1:

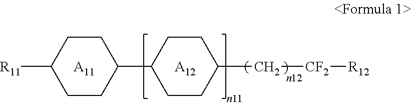

Wherein in Formula 1,

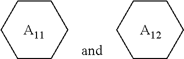

are each independently any one selected from Formulae 10-1 to 10-11 below:

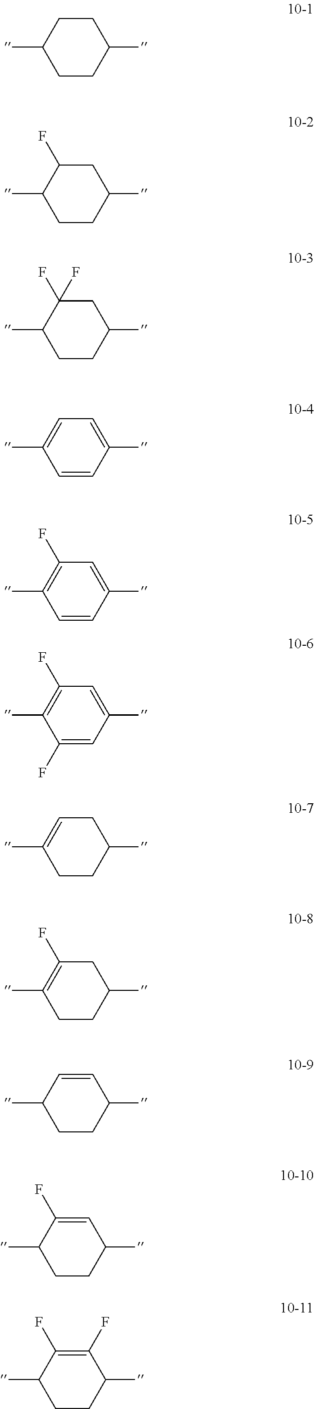

Wherein in Formulae 10-1 to 10-11, ″ may indicate a binding site to a neighboring atom; n11 may be selected from 1, 2, and 3; n12 may be selected from 0, 1, 2, 3, and 4; R₁₁ may be selected from a hydrogen, a C₁-C₅ alkyl group, a C₁-C₅ alkoxy group, and a C₂-C₅ alkenyl group; and a C₁-C₅ alkyl group substituted with —F, a C₁-C₅ alkoxy group substituted with —F, and a C₂-C₅ alkenyl group substituted with —F; and R₁₂ may be selected from a hydrogen and a C₁-C₅ alkyl group.

According to one or more embodiments of the present invention, a liquid crystal display may include a first electrode; a second electrode; and a liquid crystal layer that is disposed between the first electrode and the second electrode and and the liquid crystal layer may include the liquid crystal composition described above.

### DETAILED DESCRIPTION OF THE INVENTION

The present invention will now be described more fully with reference to exemplary embodiments. The disclosure of the present invention may, however, be embodied in many different forms and should not be construed as being limited to the embodiments set forth herein; rather, these embodiments may be provided so that this disclosure will be thorough and complete, and will fully convey the concept of the disclosure to those skilled in the art. Advantages, features, and how to achieve them of the present invention will become apparent by reference to the embodiment that will be described later in detail. This invention may, however, be embodied in many different forms and should not be limited to the exemplary embodiments.

As used herein, the terms as “first”, “second”, etc., may be used only to distinguish one component from another, and such components should not be limited by these terms.

As used herein, the singular forms “a”, “an” and “the” may be intended to include the plural forms as well, unless the context clearly indicates otherwise.

It will be further understood that the terms “comprises” and/or “comprising” used herein specify the presence of stated features or components, but do not preclude the presence or addition of one or more other features or components.

It will be understood that when a layer, region, or component is referred to as being “on” or “onto” another layer, region, or component, it may be directly or indirectly formed on the other layer, region, or component. That is, for example, intervening layers, regions, or components may be present.

As used herein, “Tni” denotes a liquid crystal operating temperature. A liquid crystal composition may undergo a nematic-to-isotropic phase transition at Tni or higher. A liquid crystal composition suitable for an active matrix liquid-crystal display (AMLCD) may have a liquid crystal operating temperature (Tni) of about 60° C. or higher to about 120° C. A liquid crystal composition suitable for an active matrix liquid-crystal display (AMLCD) may be nematic in a temperature range of about −20° C. to about 120° C.

“Δ∈” used herein may denote dielectric anisotropy. The expression “negative dielectric anisotropy” may mean that Δ∈ is less than 0.

“Δn” used herein may denote anisotropy in refractive index. A liquid crystal composition suitable for liquid crystal displays may have a Δn value of about 0.060 to 0.300. For a twisted nematic (TN), vertical alignment (VA), in-plane switching (IPS), or fringe field switching (FFS) mode LCD, the product of multiplying Δn by a cell gap (d) may be in a range of about 0.20 μm to about 0.50 μm.

“η” used herein may denote a flow viscosity. A smaller η may be better for an AMLCD. For example, a liquid crystal composition may have a flow viscosity (η) that is larger than 0 and equal to or less than 50 cP, which may be converted into a rotational viscosity (yl) that is larger than 0 and equal to or less than about 300 mPa·s.

“VHR” used herein may denote a voltage holding ratio.

A liquid crystal composition according to an embodiment of the present invention may include at least one of first liquid crystal compounds represented by Formula 1:

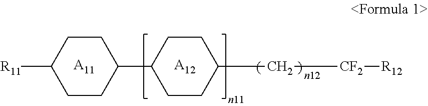

Wherein, in Formula 1,

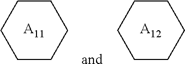

may be each independently any one selected from Formulae 10-1 to 10-11 below:

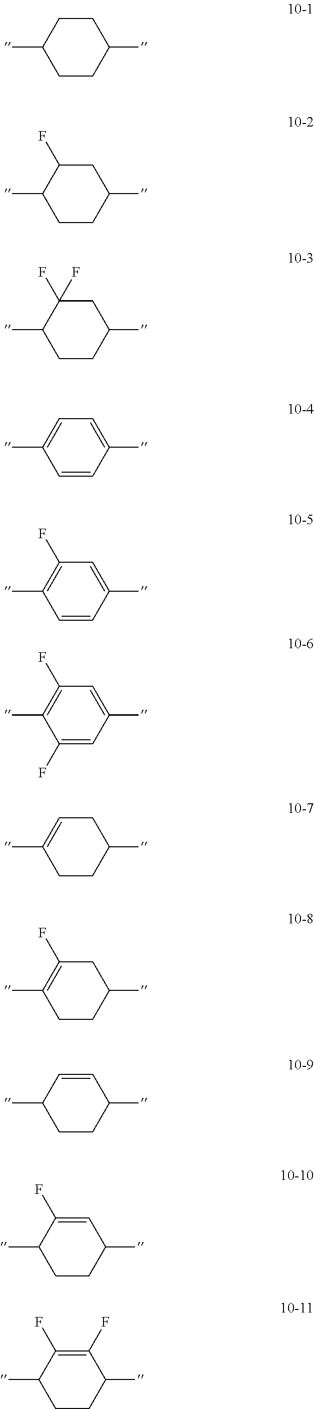

Wherein in Formulae 10-1 to 10-11, ″ may indicate a binding site to a neighboring atom.

The Formulae 10-2 and 10-3 may each independently include different optical isomers or one optical isomer.

In some embodiments,

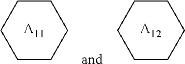

in Formula 1 may be each independently any one selected from Formulae 10-1 to 10-6, but are not limited thereto.

In some embodiments,

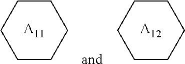

in Formula 1 may be each independently any one selected from Formulae 10-1 to 10-3, but are not limited thereto.

In some embodiments,

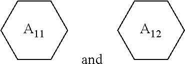

in Formula 1 may be each independently represented by Formula 10-1, but are not limited thereto.

n11 in Formula 1 indicates the number of

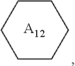

and may be selected from 1, 2, and 3. When n11 is 2 or more, a plurality of

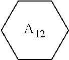

may be identical or different. For example, n11 in Formula 1 may be 1, but is not limited thereto.

n12 in Formula 1 indicates the number of CH₂ and may be selected from 0, 1, 2, 3 and 4. When n12 is 0, it means a single bond. For example, n12 in Formula 1 may be selected from 0, 1, and 2, but is not limited thereto.

R₁₁ in Formula 1 may be selected from a hydrogen, a C₁-C₅ alkyl group, a C₁-C₅ alkoxy group, and a C₂-C₅ alkenyl group; and a C₁-C₅ alkyl group substituted with —F, a C₁-C₅ alkoxy group substituted with —F, and a C₂-C₅ alkenyl group substituted with —F.

For example, R₁₁ in Formula 1 may be selected from a hydrogen, a C₁-C₅ alkyl group, a C₁-C₅ alkoxy group, and a C₂-C₅ alkenyl group, but is not limited thereto.

In some embodiments, R₁₁ in Formula 1 may be selected from a hydrogen, a methyl group, an ethyl group, a propyl group, a butyl group, a pentyl group, a methoxy group, an ethoxy group, a propoxy group, a butoxy group, a pentoxy group, an ethenyl group, a propenyl group, a butenyl group, and a pentenyl group, but is not limited thereto.

In some embodiments, R₁₁ in Formula 1 may be selected from a hydrogen, a methyl group, an ethyl group, an n-propyl group, an n-butyl group, an n-pentyl group, a methoxy group, an ethoxy group, an n-propoxy group, an n-butoxy group, an n-pentoxy group, an ethenyl group, a 1-propenyl group, a 2-propenyl group, a 1-butenyl group, a 2-butenyl group, a 1-pentenyl group, and a 2-pentenyl group, but is not limited thereto.

In some embodiments, R₁₁ in Formula 1 may be selected from a methyl group, an ethyl group, an n-propyl group, a methoxy group, an ethoxy group, an n-propoxy group, an ethenyl group, and a 1-propenyl group, but is not limited thereto.

R₁₂ in Formula 1 may be selected from a hydrogen and a C₁-C₅ alkyl group.

For example, R₁₂ in Formula 1 may be selected from a hydrogen, a methyl group, an ethyl group, a propyl group, a butyl group, and a pentyl group, but is not limited thereto.

In some embodiments, R₁₂ in Formula 1 may be selected from a hydrogen, a methyl group, an ethyl group, an n-propyl group, an n-butyl group, and an n-pentyl group, but is not limited thereto.

In some embodiments, R₁₂ in Formula 1 may be selected from a hydrogen, a methyl group, an ethyl group, and an n-propyl group, but is not limited thereto.

R₁₁ and R₁₂ in Formula 1 may be each independently substituted with the substituents described above, a formed liquid crystal composition may have low viscosity and low volatile properties.

The first liquid crystal compound may be represented by Formula 1-1 below, but is not limited thereto:

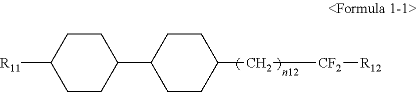

R₁₁, R₁₂, and n12 in Formula 1-1 may be the same as defined in connection with Formula 1.

The first liquid crystal compound may be represented by any one of Compounds 10 to 34 below, but is not limited thereto:

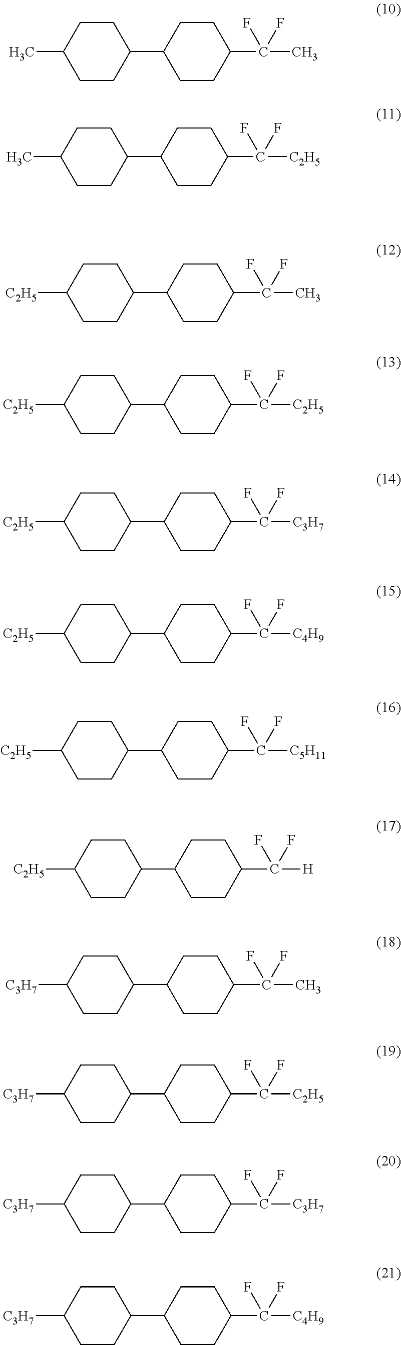

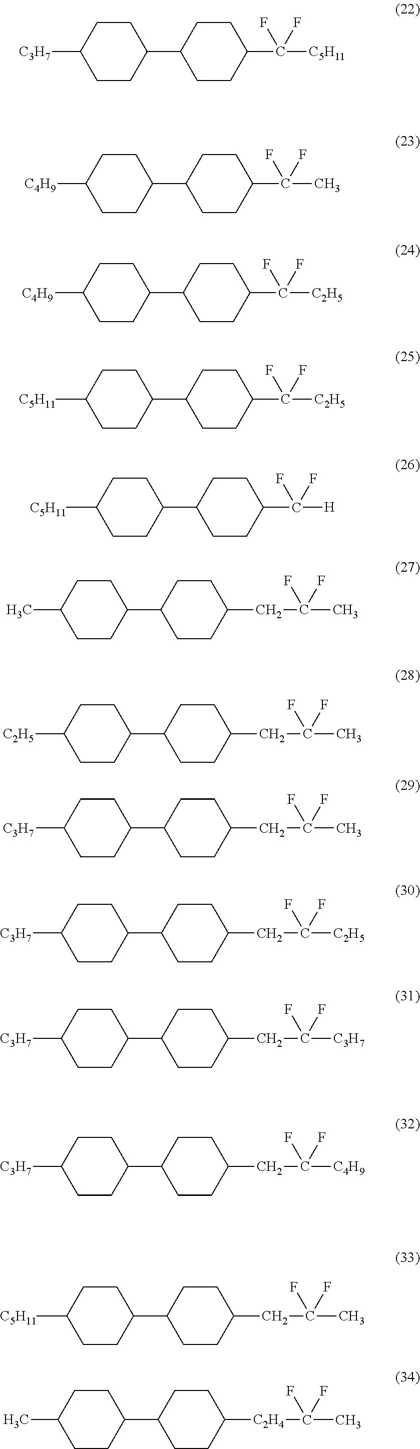

In Compounds 10 to 34, C₃H₇ may be a n-propyl group, C₄H₉ may be a n-butyl group, and C₅H₁₁ may be a n-pentyl group. However, embodiments are not limited thereto.

The liquid crystal composition may further include the first liquid crystal compound in an amount of about 0.1 to about 60 wt %. For example, an amount of the first liquid crystal compound in the liquid crystal composition may be 1 wt % or more, 5 wt % or more, or 10 wt % or more, but is not limited thereto. In some embodiments, the first liquid crystal compound in the liquid crystal composition may be 55 wt % or less, 50 wt % or less, or 45 wt % or less, but is not limited thereto. In some embodiments, the first liquid crystal compound in the liquid crystal composition may be in a range of about 5 to about 30 wt %, but the amount thereof is not limited thereto.

When the amount of first liquid crystal compound is within these ranges, a formed liquid crystal composition may have low viscosity and high specific resistance and VHR. When the amount of the first liquid crystal compound is greater than 60 wt %, Tni of the liquid crystal composition may be 60 degrees or lower. When the amount of the first liquid crystal compound is greater than 60 wt %, Δn of the liquid crystal composition may be 0.050 or lower. When the amount of the first liquid crystal compound is smaller than 0.1 wt %, the viscosity of the liquid crystal composition may increase.

The liquid crystal composition may have improved specific resistance or VHR, due to the inclusion of the first liquid crystal compound. When the liquid crystal composition includes the first liquid crystal compound, the absolute value of Δ∈ of the liquid crystal composition is high. Accordingly, a liquid crystal display having a low driving voltage and a high contrast ratio may be obtained. Due to the inclusion of the first liquid crystal compound, the liquid crystal composition may have improved stability against UV. Accordingly, the yield of the liquid crystal display including the liquid crystal composition may be increased.

The liquid crystal composition may further include at least one of second liquid crystal compounds represented by Formula 2, but is not limited thereto:

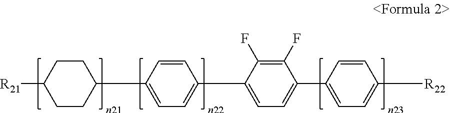

Wherein in Formula 2, R₂₁ and R₂₂ may be each independently selected from a C₁-C₇ alkyl group, a C₁-C₇ alkoxy group, and a C₂-C₇ alkenyl group; n21, n22, and n23 may be each independently selected from 0, 1, 2, and 3; and the sum of n21, n22, and n23 may be selected from 1, 2, and 3.

For example, when the liquid crystal composition includes a second liquid crystal compound that is represented by Formula 2 in which the sum of n21, n22, and n23 is 1, an amount of the second liquid crystal compound may be in a range of about 0.1 to about 40 wt %, but is not limited thereto. When the sum of n21, n22, and n23 in Formula 2 is 1, the second liquid crystal compound may have a relatively low Tni. When the sum of n21, n22, and n23 in Formula 2 is 1, the liquid crystal composition may include the second liquid crystal compound in this range of an amount, thereby providing a liquid crystal composition having a commercially available level of Tni.

In some embodiments, when the liquid crystal composition includes the second liquid crystal compound in which the sum of n21, n22, and n23 in Formula 2 is 2, the amount of the second liquid crystal compound may be in a range of about 0.1 to about 60 wt %, but is not limited thereto. When the sum of n21, n22, and n23 in Formula 2 is 2, the liquid crystal composition may include the second liquid crystal compound in this range of an amount, thereby providing a liquid crystal composition having a commercially available level of viscosity.

In some embodiments, when the liquid crystal composition includes the second liquid crystal compound in which the sum of n21, n22, and n23 in Formula 2 is 3, the amount of the second liquid crystal compound may be in a range of about 0.1 to about 30 wt %, but is not limited thereto. When the sum of n21, n22, and n23 in Formula 2 is 3, the second liquid crystal compound has a high melting point. Accordingly, when the liquid crystal composition includes the second liquid crystal compound in this range of an amount, crystalinity of the liquid crystal composition may be low.

For example, R₂₁ in Formula 2 may be a C₁-C₇ alkyl group; R₂₂ may be selected from a C₁-C₅ alkyl group and a C₁-C₅ alkoxy group, but they are not limited thereto.

In some embodiments, R₂₁ in Formula 2 may be selected from a methyl group, an ethyl group, a propyl group, a butyl group, a pentyl group, a hexyl group, and a heptyl group; and R₂₂ may be selected from a methyl group, an ethyl group, a propyl group, a butyl group, a pentyl group, a methoxy group, and an ethoxy group, but they are not limited thereto.

In some embodiments, the second liquid crystal compound may be represented by any one of Formulae 2-1 to 2-4 below, but is not limited thereto:

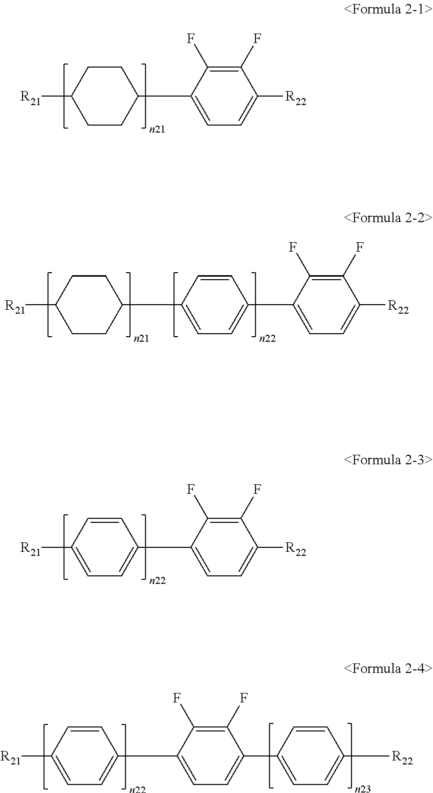

Wherein in Formulae 2-1 to 2-4, R₂₁, R₂₂, n21, n22, and n23 mat be the same as defined in connection with Formula 2.

In some embodiments, the second liquid crystal compound may be represented by any one of Compounds 35 to 77 below, but is not limited thereto:

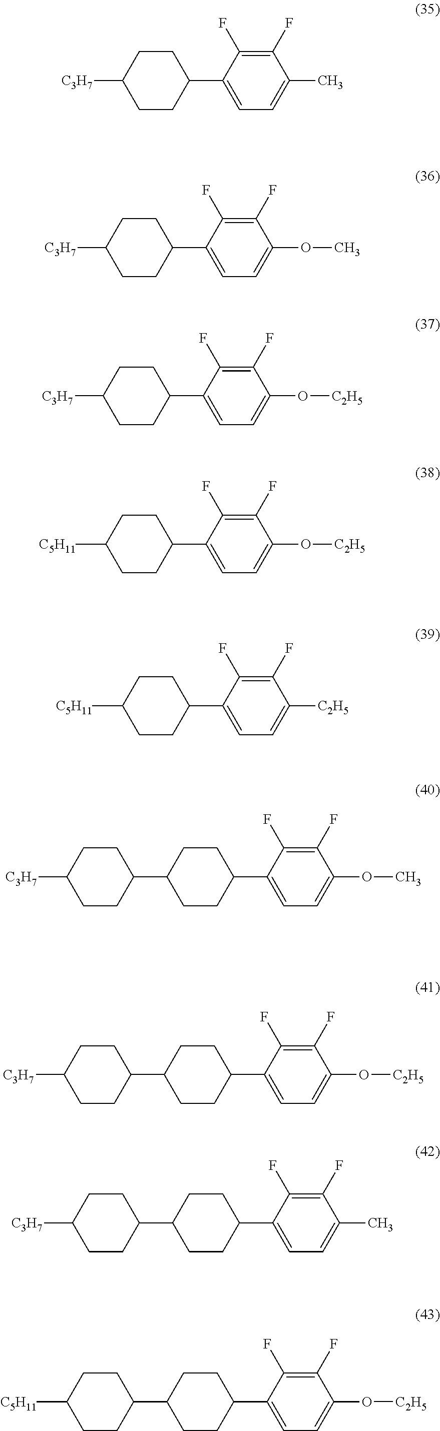

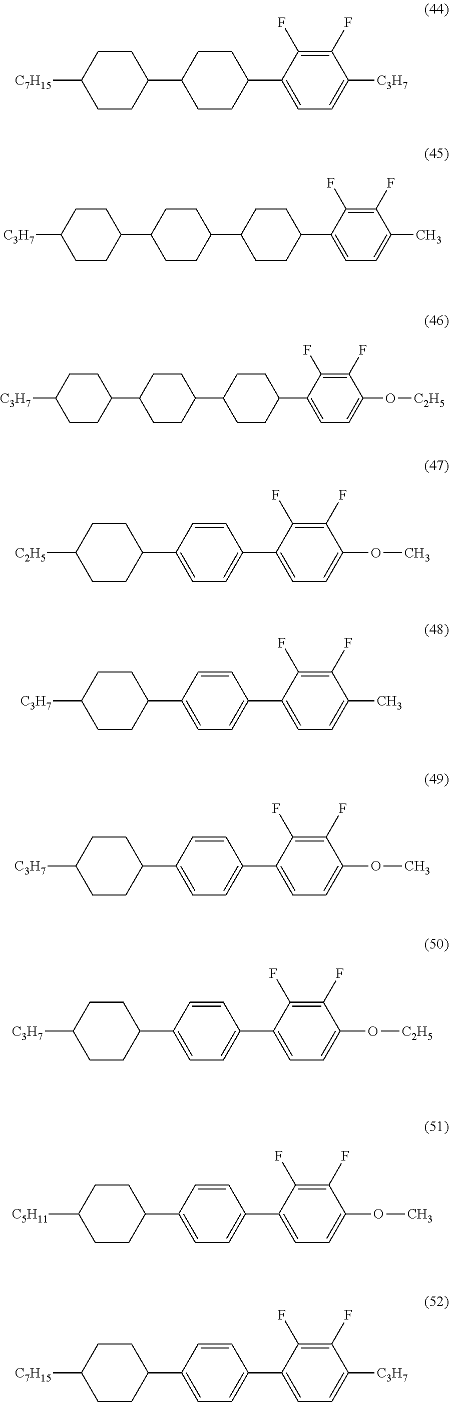

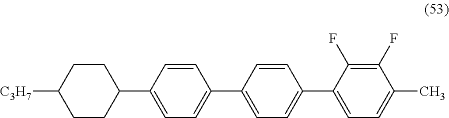

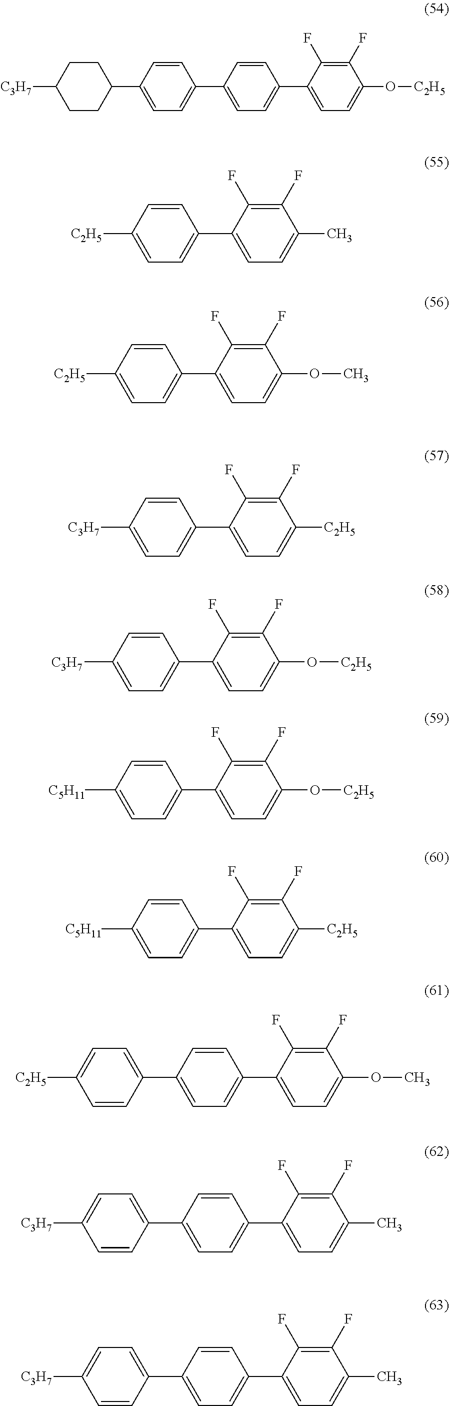

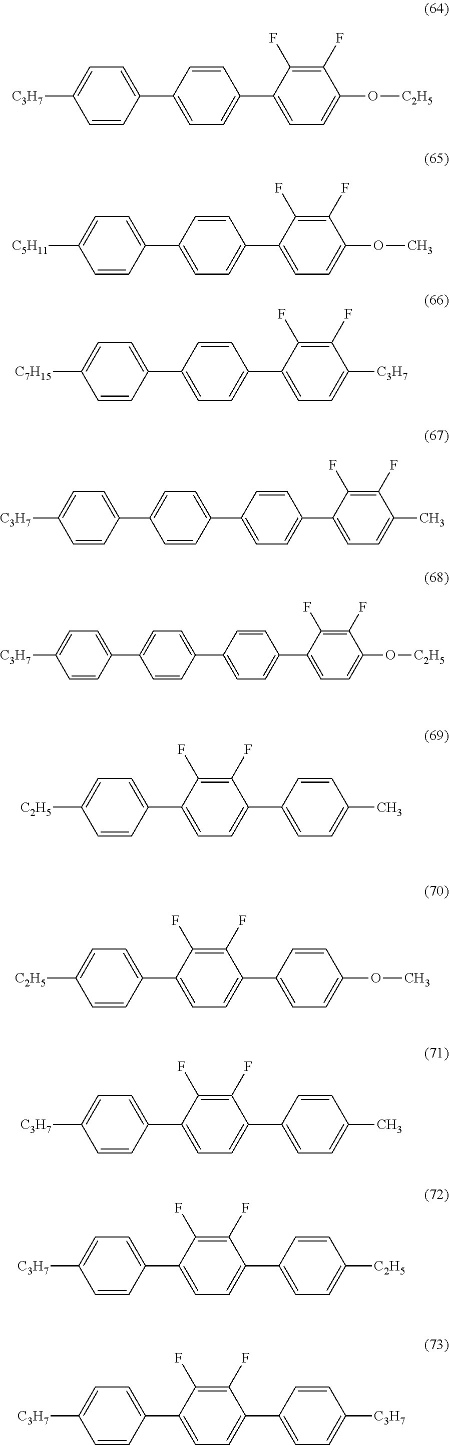

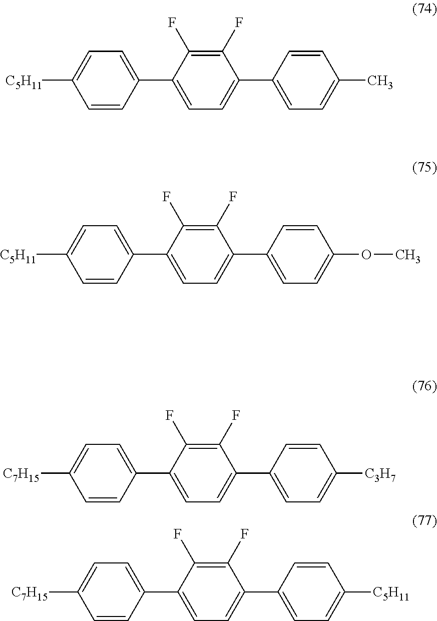

In Compounds 35 to 77, C₃H₇ may be a n-propyl group, C₄H₉ may be a n-butyl group, C₅H₁₁ may be a n-pentyl group, C₆H₁₃ may be a n-hexyl group, C₇H₁₅ may be a n-heptyl group, but they are not limited thereto.

In some embodiments, the liquid crystal composition may further include at least one of third liquid crystal compounds represented by Formula 3, but is not limited thereto:

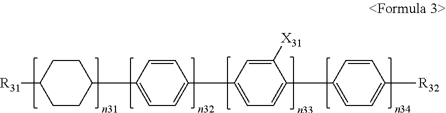

Wherein in Formula 3, R₃₁ and R₃₂ may be each independently selected from a C₁-C₇ alkyl group, a C₁-C₇ alkoxy group, and a C₂-C₇ alkenyl group; X₃₁ may be selected from a hydrogen and —F; n31, n32, n33, and n34 may be each independently selected from 0, 1, 2 and 3; the sum of n31, n32, n33, and n34 may be selected from 2, 3, and 4.

In some embodiments, when the liquid crystal composition includes the third liquid crystal compound in which the sum of n31, n32, n33, and n34 in Formula 3 is 2, the amount of the third liquid crystal compound may be in a range of about 0.1 to about 50 wt %, but is not limited thereto. When the sum of n31, n32, n33, and n34 in Formula 3 is 2, the liquid crystal composition may include the third liquid crystal compound in the range described above, thereby providing a liquid crystal composition having a commercially available level of Tni.

In some embodiments, when the liquid crystal composition includes the third liquid crystal compound in which the sum of n31, n32, n33, and n34 in Formula 3 is 3, the amount of the third liquid crystal compound may be in a range of about 0.1 to about 50 wt %, but is not limited thereto. When the sum of n31, n32, n33, and n34 in Formula 3 is 3, the liquid crystal composition may include the third liquid crystal compound in the range described above, thereby providing a liquid crystal composition having a commercially available level of viscosity.

In some embodiments, when the liquid crystal composition includes the third liquid crystal compound in which the sum of n31, n32, n33, and n34 in Formula 3 is 4, the amount of the third liquid crystal compound may be in a range of about 0.1 to about 30 wt %, but is not limited thereto. When the sum of n31, n32, n33, and n34 is 4, the third liquid crystal compound has a high melting point. Accordingly, when the liquid crystal composition includes the third liquid crystal compound in this range of the amount, crystalinity of the liquid crystal composition may be low.

For example, in Formula 3, R₃₁ may be selected from a C₁-C₇ alkyl group and a C₂-C₇ alkenyl group; R₃₂ may be selected from a hydrogen, a C₁-C₅ alkyl group, a C₁-C₅ alkoxy group, and a C₂-C₅ alkenyl group, but they are not limited thereto.

In some embodiments, R₃₁ in Formula 3 may be selected from a methyl group, an ethyl group, a propyl group, a butyl group, a pentyl group, a hexyl group, a heptyl group, and an ethenyl group; and R₃₂ may be selected from a methyl group, an ethyl group, a propyl group, a butyl group, a pentyl group, a methoxy group, an ethoxy group, an n-propoxy group, an ethenyl group, and a propenyl group, but is not limited thereto.

In some embodiments, the third liquid crystal compound may be represented by any one of Formulae 3-1 to 3-3 below, but is not limited thereto:

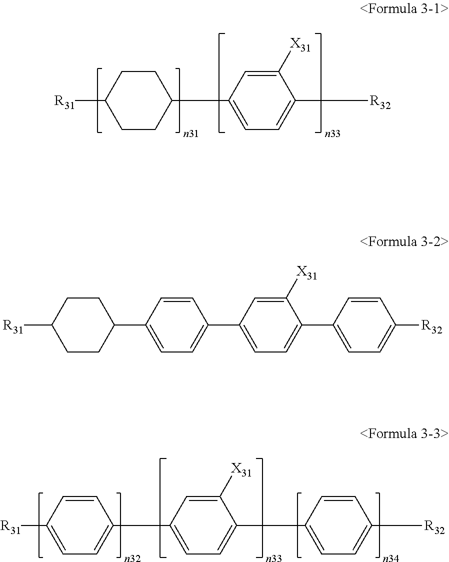

Wherein in Formulae 3-1 to 3-3, R₃₁, R₃₂, X₃₁, n31, n32, n33, and n34 may be the same as defined in connection with Formula 3.

In some embodiments, the third liquid crystal compound may be represented by any one of Compounds 78 to 119 below, but is not limited thereto:

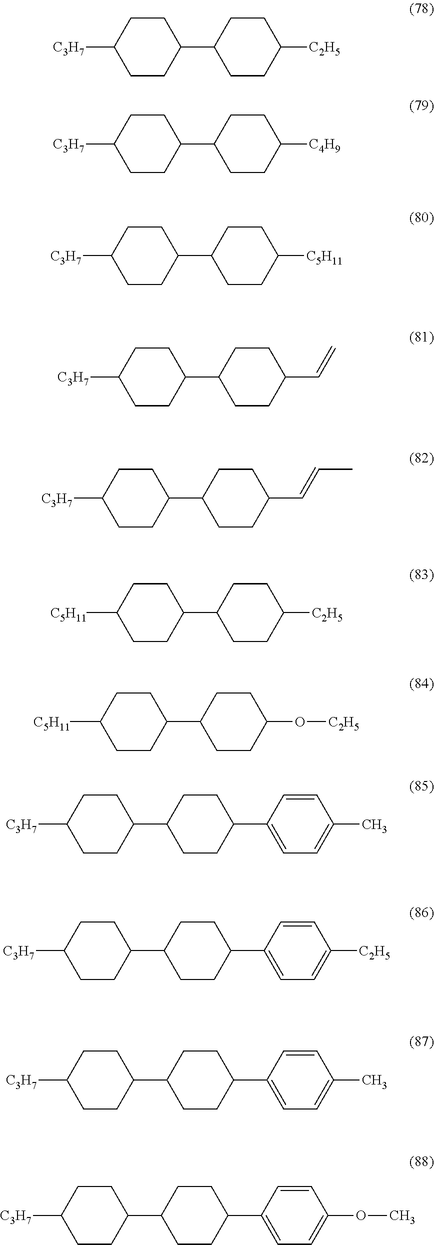

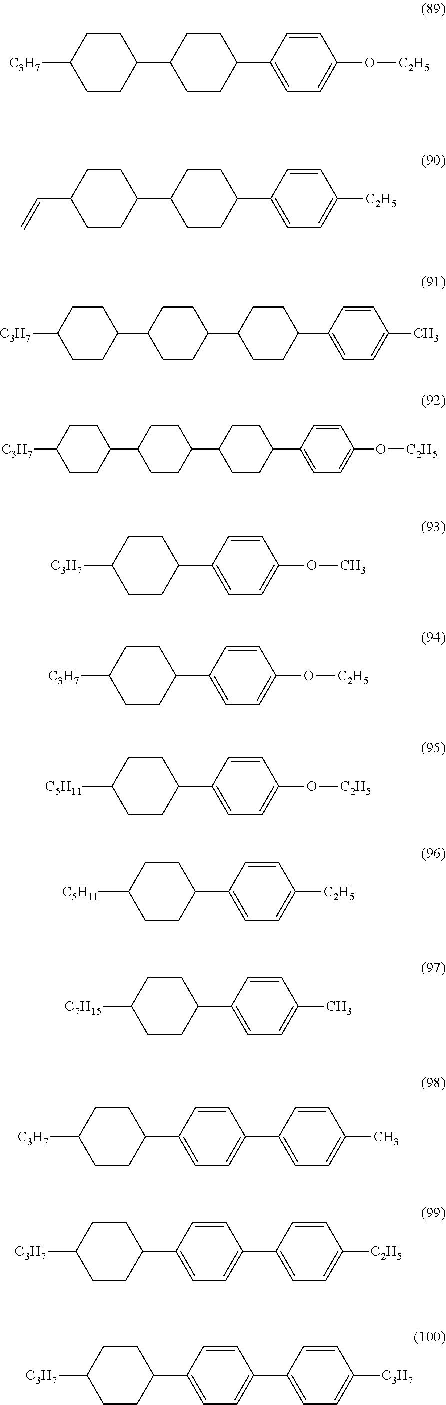

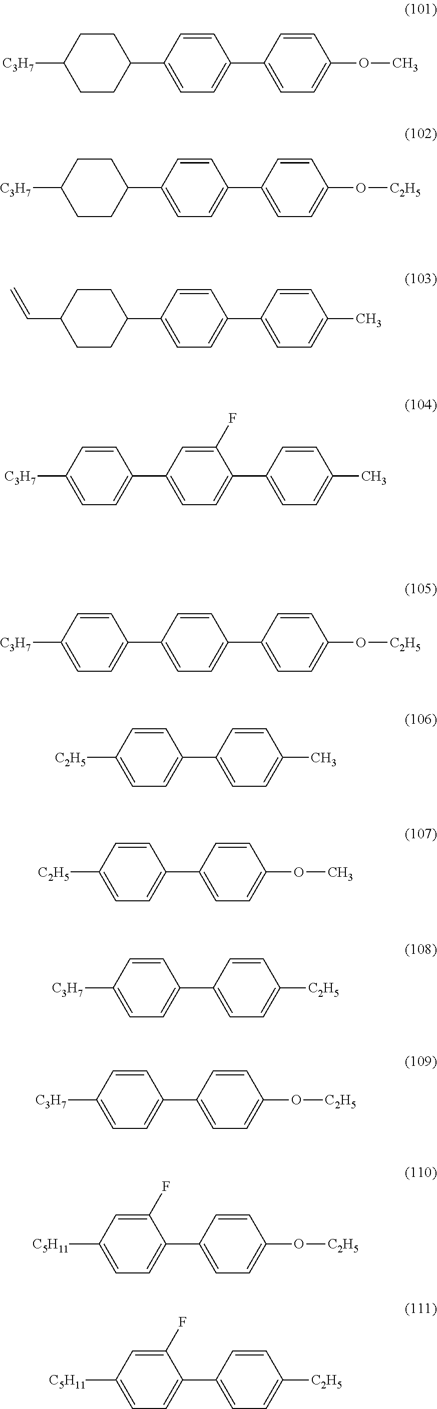

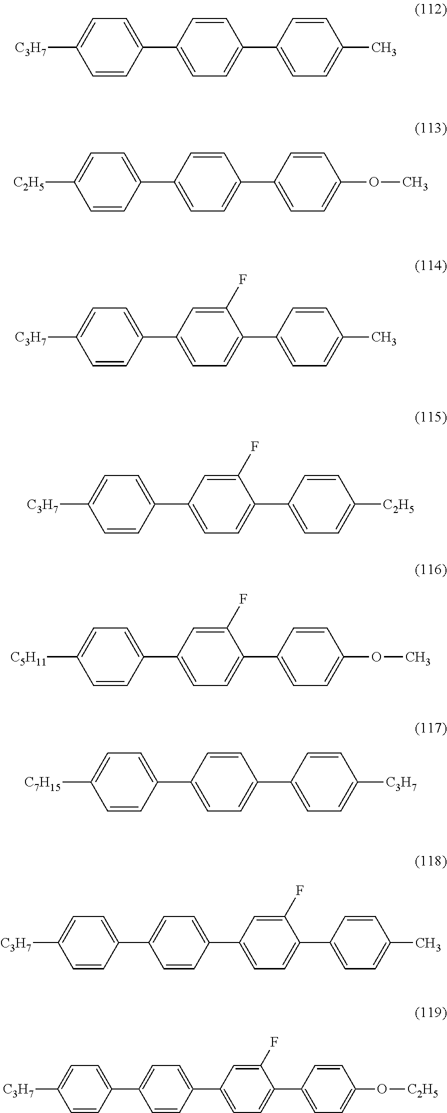

In Compounds 78 to 119, C₃H₇ may be a n-propyl group, C₅H₁₁ may be a pentyl group, and C₇H₁₅ may be a n-heptyl group. However, embodiments are not limited thereto.

Regarding the third liquid crystal compound, an absolute value of Δ∈ is almost 0. The third liquid crystal compound may be included in the liquid crystal composition to control Δn.

In some embodiments, the liquid crystal composition may further include at least one of fourth liquid crystal compounds represented by Formula 4, but is not limited thereto:

<Formula 4>

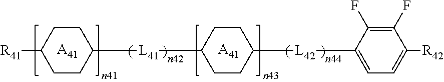

Wherein in Formula 4,

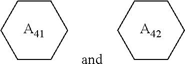

may be each independently any one selected from Formulae 10-1 to 10-11 below;

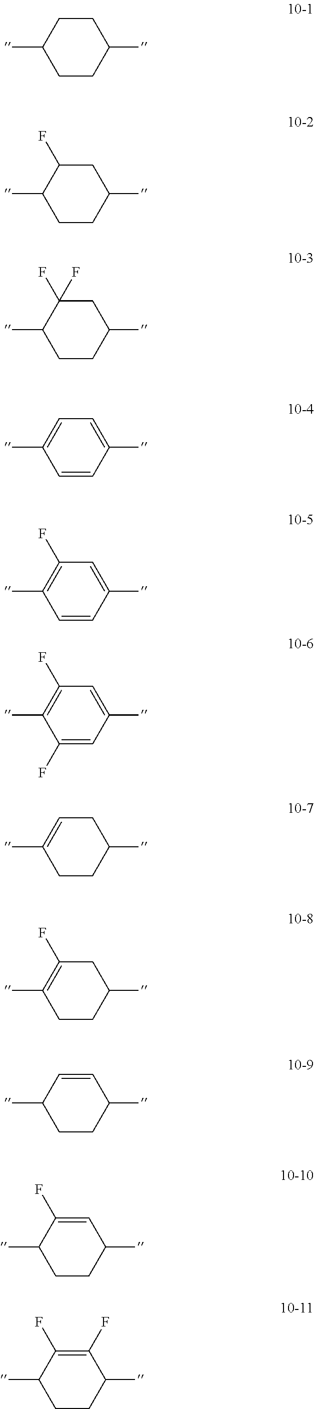

Wherein in Formula 4, L₄₁ and L₄₂ may be each independently selected from —CH₂CH₂—, —CH₂O—, —OCH₂—, —CF₂O—, and —OCF₂—; R₄₁ and R₄₂ may be each independently selected from a C₁-C₇ alkyl group, a C₁-C₇ alkoxy group, and a C₂-C₇ alkenyl group; n41 may be selected from 1, 2, and 3; n42 to n44 may be each independently selected from 0, 1, and 2; the sum of n42 and n44 may be selected from 1 and 2; and ″ in the formula above indicates a binding site to a neighboring atom.

In some embodiments, when the liquid crystal composition includes the fourth liquid crystal compound in which the sum of n41 and n43 in Formula 4 is 1, the amount of the fourth liquid crystal compound may be in a range of about 0.1 to about 30 wt %, but is not limited thereto. When the sum of n41 and n43 in Formula 4 is 1, the liquid crystal composition may include the fourth liquid crystal compound in the range described above, thereby providing a liquid crystal composition having a commercially available level of Tni.

In some embodiments, when the liquid crystal composition includes the fourth liquid crystal compound in which the sum of n41 and n43 in Formula 4 is 2, the amount of the fourth liquid crystal compound may be in a range of about 0.1 to about 50 wt %, but is not limited thereto. When the sum of n41 and n43 in Formula 4 is 2, the liquid crystal composition may include the fourth liquid crystal compound in the range described above, thereby providing a liquid crystal composition having a commercially available level of viscosity.

In some embodiments, when the liquid crystal composition includes the fourth liquid crystal compound in which the sum of n41 and n43 in Formula 4 is 3, the amount of the fourth liquid crystal compound may be in a range of about 0.1 to about 30 wt %, but is not limited thereto. When the sum of n41 and n43 in Formula 4 is 3, the fourth liquid crystal compound has a high melting point. Accordingly, when the liquid crystal composition includes the fourth liquid crystal compound in this range of an amount, crystalinity of the liquid crystal composition may be low.

In some embodiments,

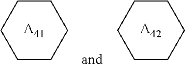

in Formula 4 may be each independently any one selected from Formulae 10-1 and 10-6, but are not limited thereto.

In some embodiments, when the liquid crystal composition includes the fourth liquid crystal compound in which the sum of n41 and n43 in Formula 4 is 3, the amount of the fourth liquid crystal compound may be in a range of about 0.1 to about 30 wt %, but is not limited thereto. When the sum of n41 and n43 in Formula 4 is 3, the fourth liquid crystal compound has a high melting point. Accordingly, when the liquid crystal composition includes the fourth liquid crystal compound in this range of an amount, crystalinity of the liquid crystal composition may be low.

In some embodiments, L₄₁ and L₄₂ in Formula 4 may be selected from —CH₂CH₂—, —CH₂O—, and —CF₂O—, but they are not limited thereto.

In some embodiments, R₄₁ in Formula 4 may be a C₁-C₅ alkyl group; R₄₂ may be selected from a C₁-C₅ alkyl group and a C₁-C₅ alkoxy group, but they are not limited thereto.

In some embodiments, in Formula 4, R₄₁ may be selected from a methyl group, an ethyl group, a propyl group, a butyl group, and a pentyl group; and R₄₂ may be selected from a methyl group, an ethyl group, a n-propyl group, a methoxy group, an ethoxy group, and a n-propoxy group, but they are not limited thereto.

In some embodiments, the fourth liquid crystal compound may be represented by Formula 4-1 below, but is not limited thereto:

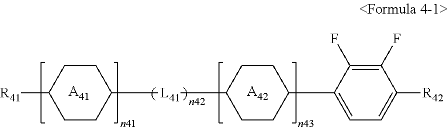

Wherein in Formula 4-1, A₄₁, A₄₂, L₄₁, L₄₂, R₄₁, R₄₂, and n41 to n44 may be the same as defined in connection with Formula 4.

In some embodiments, the fourth liquid crystal compound may be represented by any one of Compounds 120 to 134 below, but is not limited thereto:

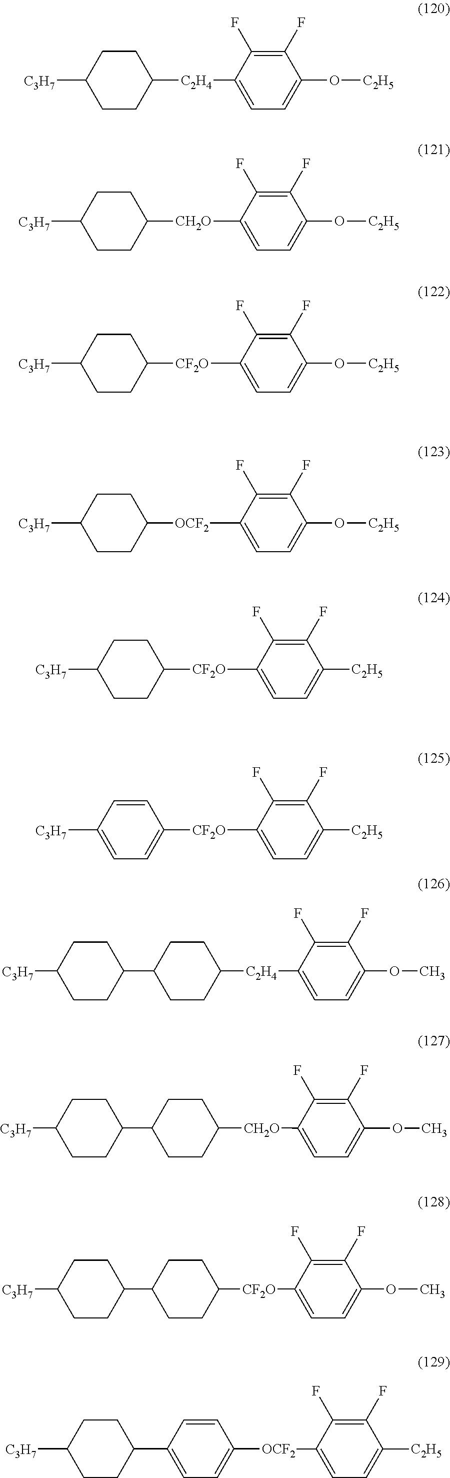

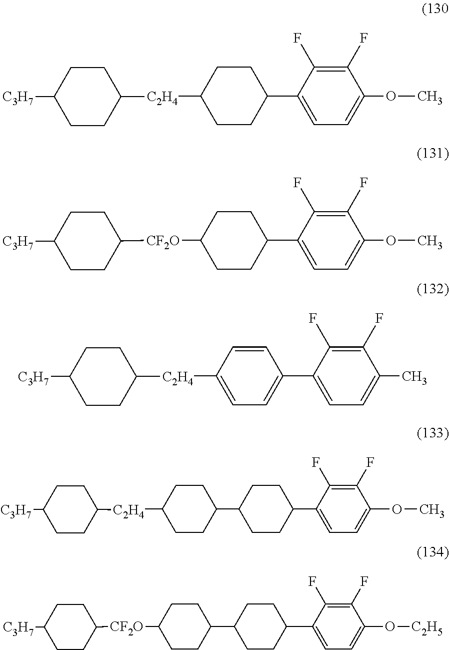

The first liquid crystal compound, the second liquid crystal compound, the third liquid crystal compound, and the fourth liquid crystal compound may be obtained by using a known organic synthesis method.

For example, the first liquid crystal compound may be synthesized by using a method disclosed in Japanese Patent Publication No. 1994-192142.

The liquid crystal composition may be prepared by using a commercially available method. For example, the liquid crystal composition may be prepared by dissolving liquid crystal compounds at high temperature. In some embodiments, the liquid crystal composition may be prepared by mixing such liquid crystal compounds as described above with an organic solvent such as acetone, chloroform, or methanol, and then removing the organic solvent therefrom.

The liquid crystal composition may further include an appropriate additive if required. For example, the liquid crystal composition may further include at least one selected from a chiral agent, a polymerizable additive, and a stabilizing agent. However, embodiments are not limited thereto.

The chiral agent may improve orientation of the liquid crystal compound. When the liquid crystal composition further includes a chiral agent, the liquid crystal composition may be an electrically controlled birefringence (ECB) mode liquid crystal composition or a dynamic scattering (DS) mode liquid crystal composition.

In the liquid crystal composition, the amount of the chiral agent may be in a range of about 0.01 wt % to about 30 wt %, in consideration of preparation costs of the liquid crystal composition, but is not limited thereto.

Non-limiting examples of the chiral agent may be the compounds represented by the following formulae:

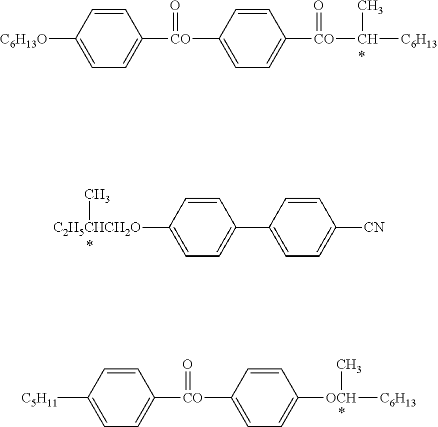

For example, the chiral agent may be selected from Compounds 135 to 142 but is not limited thereto:

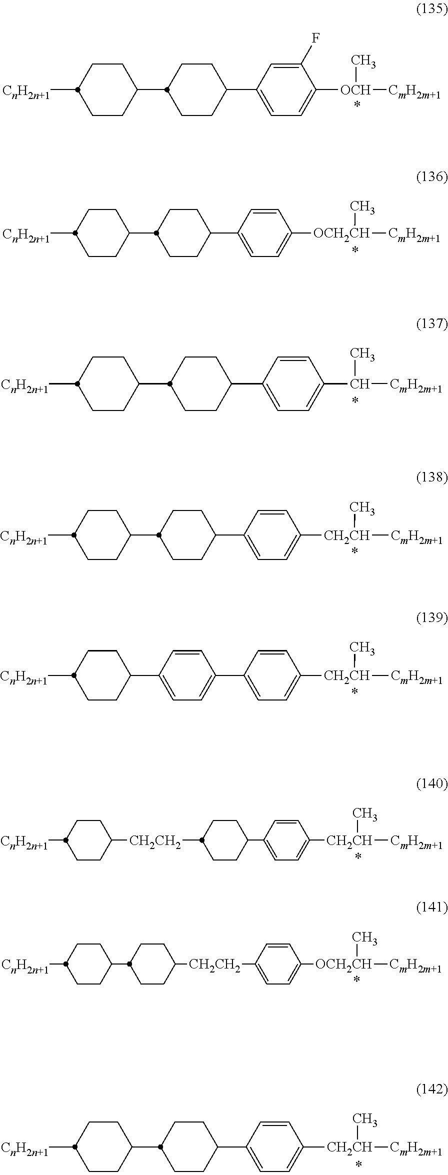

In Compounds 135 to 142, m may be selected from 2, 3, 4, 5, 6, 7, 8, 9, 10, 11, and 12, and n may be selected from 1, 2, 3, 4, 5, 6, 7, 8, 9, 10, 11, and 12. For example, in Compounds 135 to 142, m may be selected from 2, 3, 4, 5, 6, 7, 8, 9, and 10, and n may be selected from 1, 2, 3, 4, 5, 6, 7, 8, 9, and 10, but they are not limited thereto.

In the liquid crystal composition, the amount of the polymerizable additive may be in a range of about 0.01 wt % to about 20 wt %, but is not limited thereto. When the liquid crystal composition further includes a polymerizable additive, the liquid crystal composition may be used in a liquid crystal display that controls alignment of liquid crystal molecules through light irradiation. An example of such a liquid crystal display is a polymer stabilized vertical alignment (PSVA) mode liquid crystal display.

For example, the polymerizable additive may be Compound 143, but is not limited thereto:

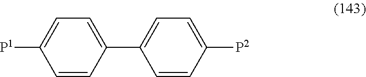

Wherein P¹ and P² in Compound 143 may be each independently selected from an acrylate group, a methacrylate group, a vinyl group, a vinyloxy group, and an epoxy group.

For example, P¹ and P² in Formula 143 may be each independently selected from an acrylate group and a methacrylate group, but they are not limited thereto.

In the liquid crystal composition, the amount of the stabilizing agent may be in a range of about 0.0001 wt % to about 5 wt %, but is not limited thereto. For example, the amount of the stabilizing agent may be in a range of about 0.001 wt % to about 1 wt %, but is not limited thereto.

For example, the stabilizing agent may be an antioxidant, but is not limited thereto. Non-limiting examples of the antioxidant may be Compounds 144 to 149 below:

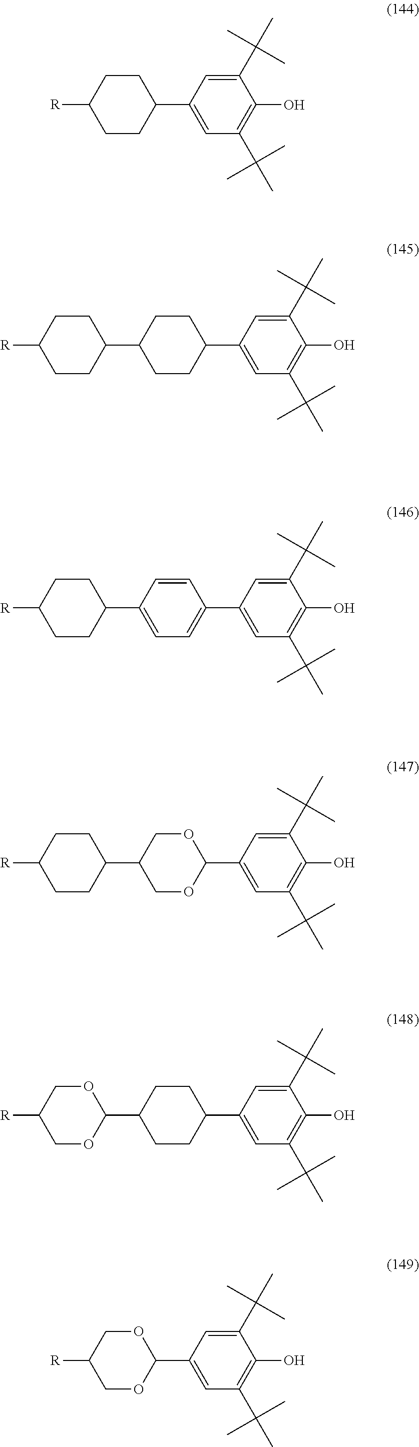

R in Compounds 144 to 149 may be selected from a hydrogen, a C₁-C₁₈ alkyl group, a C₁-C₁₈ alkoxy group, and a C₂-C₁₈ alkenyl group; and a C₁-C₁₈ alkyl group, a C₁-C₁₈ alkoxy group, and a C₂-C₁₈ alkenyl group, each substituted with at least one selected from a halogen atom and a cyano group.

For example, the stabilizing agent may be a hindered amine light stabilizer (HALS) available from ADEKA Corporation, but is not limited thereto. Non-limiting examples of the HALS may be LA-52 (tetrakis(1,2,2,6,6-pentamethyl-4-piperidyl) butane-1,2,3,4-tetracarboxylate), LA-57 (tetrakis(2,2,6,6-tetramethyl-4-piperidyl) butane-1,2,3,4-tetracarboxylate), and LA-72 (bis(1,2,2,6,6-pentamethyl-4-piperidyl) sebacate).

The liquid crystal composition may further include a liquid crystal compound with positive dielectric anisotropy, unless the liquid crystal compound with positive dielectric anisotropy damages desired physical properties of the liquid crystal composition.

The liquid crystal composition may be suitable for an AMLCD, but is not limited thereto. The liquid crystal composition may have a negative dielectric anisotropy.

A liquid crystal display according to an embodiment of the present invention may include a first electrode; a second electrode; and a liquid crystal layer that is disposed between the first electrode and the second electrode and the liquid crystal layer may include a liquid crystal composition as described above.

The liquid crystal display may be an in-plane switching (IPS) mode liquid crystal display, a fringe field switching (FFS) mode liquid crystal display, a plane-to-line switching (PLS) mode liquid crystal display, a vertical alignment (VA) mode liquid crystal display, a stabilized vertical alignment (SVA) mode liquid crystal display, or a polymer stabilized vertical alignment (PSVA) mode liquid crystal display, but is not limited thereto.

The liquid crystal display may further include at least one selected from an alignment layer between the first electrode and the liquid crystal layer and an alignment layer between the liquid crystal layer and the second electrode.

A liquid crystal display according to an embodiment of the present invention may include a liquid crystal composition. Due to the inclusion of the liquid crystal composition, the liquid crystal display may have an appropriate negative dielectric constant, a low viscosity, a high specific resistance, and a high VHR.

A C₁-C₇ alkyl group used herein may refer to a linear or branched aliphatic hydrocarbon monovalent group having 1 to 7 carbon atoms, and detailed examples thereof may be a methyl group, an ethyl group, a propyl group, an iso-butyl group, a sec-butyl group, a ter-butyl group, an n-pentyl group, and a hexyl group.

A C₁-C₇ alkoxy group used herein may refer to a monovalent group represented by —OA₁₀₁ (wherein A₁₀₁ is the C₁-C₇ alkyl group), and detailed examples thereof may be a methoxy group, an ethoxy group, and an iso-propyloxy group.

A C₂-C₁₈ alkenyl group used herein may refer to a hydrocarbon group formed by substituting at least one carbon double bond in the middle or terminal of the C₂-C₁₈ alkyl group, and detailed examples thereof may be an ethenyl group, a propenyl group, and a butenyl group.

Hereinafter, a liquid crystal display according to an embodiment of the present invention will be described in detail with reference to Examples. However, the liquid crystal display is not limited thereto.

### EXAMPLE

### Synthesis Example 1

### Synthesis of Compound 13

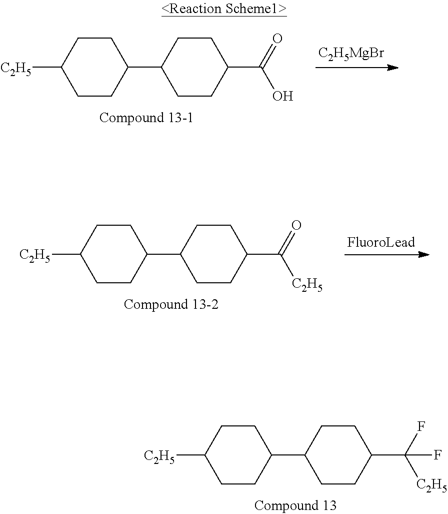 [SMILES: "C2H5MgBr" ; CCC1CCC(C2CCC(C(=O)O)CC2)CC1 ; CCC(=O)C1CCC(C2CCC(CC)CC2)CC1 ; "FluoroLead" ; CCC1CCC(C2CCC(C(F)(F)CC)CC2)CC1]

A liquid crystal compound according to an embodiment of the present invention was synthesized according to Reaction Scheme 1. First, a starting material was synthesized, and then, alkylated using a Grignard reagent, and fluorinated using FluoroLead, thereby completing the preparation of Compound 13. However, a method for the preparation of the liquid crystal compound according to an embodiment of the present invention is not limited to the synthesis method illustrated in Reaction Scheme 1. For example, the liquid crystal compound according to an embodiment of the present invention may be synthesized by referring to Mol. cryst. Liq. Cryst. 1995, Vol. 260, PP. 277-286, or by using other fluorinating reagents.

### Synthesis Example 2

### Synthesis of Compound (29)

Compound (29) was synthesized in the same manner as in Synthesis Example 1, except that Compound 29-1 was used instead of Compound 13-1.

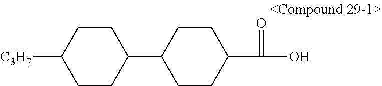

A purity of Compound (29) was confirmed as 99.29% by GC, and a specific resistance of Compound 29 dissolved in an amount of 15 wt % in an organic solvent was 7.5×10¹³ Ω·cm. A phase transition temperature of Compound 29 was measured by differential scanning calorimetry (DSC), and a melting point thereof was 20.2° C., and a temperature thereof at which a smatic-to-isotropic phase transition occurs was 98.9° C.

### Examples 1 to 5 and Comparative Example 1

Liquid crystal compositions were prepared according to Examples 1 to 5 and Comparative Example 1 by mixing such liquid crystal compounds shown in Table 1 in such amounts shown in Table 1:

TABLE 1

| | Liquid crystal compound | Amount (wt %) |
|---|---|---|
| Example 1 | Compound 36 | 18 |
| | Compound 37 | 18 |
| | Compound 41 | 22 |
| | Compound 43 | 22 |
| | Compound 13 | 20 |
| Example 2 | Compound 36 | 15 |
| | Compound 37 | 18 |
| | Compound 41 | 22 |
| | Compound 43 | 18 |
| | Compound 85 | 7 |
| | Compound 13 | 20 |
| Example 3 | Compound 37 | 15 |
| | Compound 41 | 15 |
| | Compound 42 | 10 |
| | Compound 43 | 13 |
| | Compound 78 | 15 |
| | Compound 81 | 10 |
| | Compound 85 | 7 |
| | Compound 13 | 15 |
| Example 4 | Compound 41 | 15 |
| | Compound 49 | 10 |
| | Compound 55 | 15 |
| | Compound 78 | 10 |
| | Compound 115 | 10 |
| | Compound 126 | 10 |
| | Compound 12 | 15 |
| | Compound 13 | 15 |
| Example 5 | Compound 41 | 8 |
| | Compound 49 | 15 |
| | Compound 55 | 15 |
| | Compound 115 | 15 |
| | Compound 127 | 15 |
| | Compound 10 | 15 |
| | Compound 13 | 10 |
| | Compound 20 | 7 |
| Comparative | Compound 36 | 18 |
| Example 1 | Compound 37 | 18 |
| | Compound 41 | 22 |
| | Compound 43 | 22 |
| | Compound 81 | 20 |

### Evaluation Example 2

Each of the liquid crystal compositions of Examples 1 to 5 and Comparative Example 1 was injected into a vertically aligned liquid crystal cell to evaluate a liquid crystal operating temperature Tni, a dielectric anisotropy value Δ∈, a refractive anisotropy value (Δn), a viscosity (η), and a VHR thereof according to the methods described in Table 2. Results thereof are shown in Table 3.

TABLE 2

| | |
|---|---|
| Tni | Tni was measured using a polarizing microscope equipped |
| | with a hot stage while a heating/cooling rate was |
| | maintained at about 2° C./min. |
| Δ∈ | A homeotropically aligned liquid crystal cell and a |
| | homogeneously aligned liquid crystal cell were manufactured |
| | using each of the liquid crystal compositions at 20° C. and |
| | 60 Hz by using a system (Model 6254, available from Toyo |
| | Co.) to determine ∈∥ (dielectric constant in a |
| | direction parallel to the symmetry axis) and ∈⊥ (dielectric |
| | constant in a direction perpendicular to the symmetry axis). |
| | Δ∈ was calculated using the equation (Δ∈ = ∈∥ − ∈⊥) |
| Δn | Anisotropy in refractive indexe(Δn) of liquid crystals to |
| | normal light and abnormal light were measured at 20° C. |
| | using an Abbe refractometer and an interference filter (589 |
| | nm) |
| η | A viscosity (η) was measured at 25° C. using an E-type |
| | viscometer |
| VHR | A VHR was measured using a 1-inch vertically aligned (VA) |
| (60° C.) | test cell with a VA layer (available from JSR Corporation) |
| | on an ITO substrate by an area method at 60° C. with a |
| | holding time of about 16.6 msec. |
| VHR after | A VHR was measured after radiation of UV light (15J) onto a |
| UV | 1-inch VA test cell with a VA layer (available from JSR |
| irradiation | Corporation) on an ITO substrate, by an area method at 60° C. |
| (60° C.) | with a holding time of about 16.6 msec. |

TABLE 3

| | | | | | | Compar- |
|---|---|---|---|---|---|---|
| | | | | | | ative |
| | Exam- | Exam- | Exam- | Exam- | Exam- | Exam- |
| | ple 1 | ple 2 | ple 3 | ple 4 | ple 5 | ple 1 |
| Tni (° C.) | 72 | 78 | 85 | 78 | 76 | 78 |
| Δ∈(25° C.) | −5.2 | −4.8 | −3.1 | −3.2 | −3.6 | −4.8 |
| Δn(25° C.) | 0.082 | 0.083 | 0.072 | 0.103 | 0.133 | 0.086 |
| Viscosity | 24 | 22 | 18 | 18 | 17 | 23 |
| (mPa · s) | | | | | | |
| (25° C.) | | | | | | |
| VHR (%) | 99.1 | 99.3 | 98.2 | 99.2 | 98.9 | 96.5 |
| VHR after | 98.1 | 98.4 | 96.4 | 97.1 | 96.5 | 82.5 |
| UV irradi- | | | | | | |
| ation (%) | | | | | | |

Referring to Table 3, the liquid crystal compositions of Examples 1 to 5 were confirmed to have higher reliability than the liquid crystal composition of Comparative Example 1. In particular, the liquid crystal compositions of Examples 3 to 7 have low viscosity and high reliability, thereby providing a practical and stable liquid crystal display.

### Example 6

Compound 143 was added to the liquid crystal composition of Example 4 to be about 0.3 wt % of a final mixture composition, followed by UV irradiation to thereby manufacture a 55-inch PSVA mode TV having a pretilted angle of about 88°.

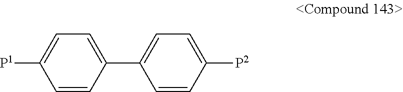

Wherein, in Compound 143, P¹ and P² are an acrylate group.

### Evaluation Example 3

A response rate of the liquid crystal composition of Example 6 was evaluated at an operating voltage of about 8 V with a cell gap of about 3.3 μm. The response rate of the liquid crystal composition was determined by measuring a rising time (Tₒₙ) and a falling time (T_{off}). As a result, the liquid crystal composition of Example 6 was confirmed to have a response rate (Tₒₙ+T_{off}) of about 8.1 ms. In consideration of the response speed of Example 6, it was conformed that a liquid crystal composition according to embodiments of the present invention provided a considerably high response rate.

### Example 7

An 8-inch FFS mode TABLET panel was manufactured using the liquid crystal composition of Example 4. The panel had a cell gap of about 3.2 μm, pixels per inch of about 200 ppi, and a driving voltage of about 4.5 V.

### Evaluation Example 4

A VHR and afterimages of the panel of Example 6 were evaluated. The VHR measurement was performed using a VHR measurement system (available from Toyo Technica) at about 25° C. with a square wave voltage of about ±5V, a pulse interval of about 60μ seconds, and a frame time of about 16.7 seconds at 30 Hz per frame. The afterimage evaluation was performed by observing afterimages displayed on a display region after applying a checker flag on the display region at about 50° C. for about 168 hours.

The obtained panel had higher VHR and less afterimages than a liquid crystal display having a positive dielectric anisotropy.

As described above, according to the one or more of the above embodiments of the present disclosure, a liquid crystal composition may have a low viscosity, an appropriate refractive anisotropy, a negative dielectric anisotropy, and stability against UV or heat.

While the inventive concept has been particularly shown and described with reference to exemplary embodiments thereof, it will be understood by those of ordinary skill in the art that various changes in form and details may be made therein without departing from the spirit and scope as defined by the following claims.

What is claimed is:

1. A liquid crystal composition comprises at least one of first liquid crystal compounds represented by Formula 1: 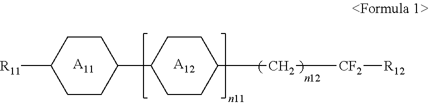
wherein in Formula 1, 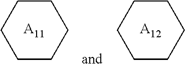
are each independently any one selected from Formulae 10-1 to 10-11 below: 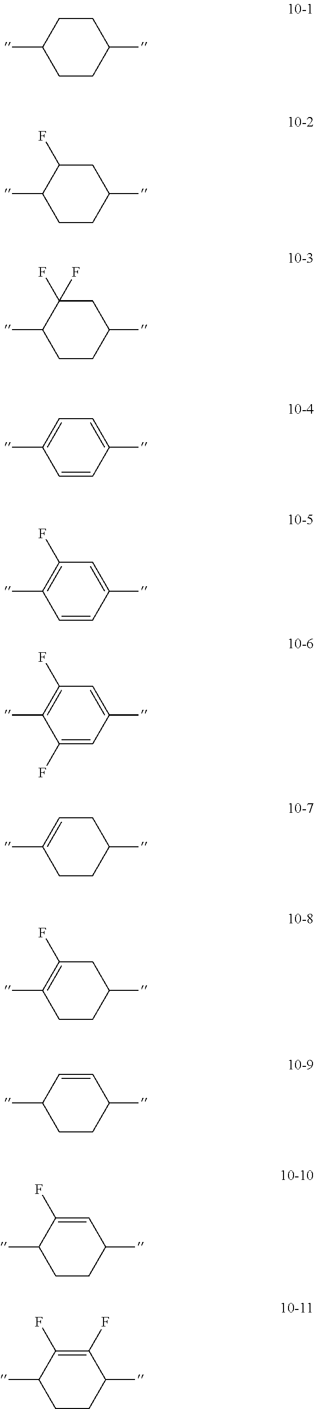
wherein in Formulae 10-1 to 10-11, ″ indicates a binding site to a neighboring atom.
n11 is selected from 1, 2, and 3;
n12 is selected from 0, 1, 2, 3, and 4;
R₁₁ is selected from a hydrogen, a C₁-C₅ alkyl group, a C₁-C₅ alkoxy group, a C₂-C₅ alkenyl group, a C₁-C₅ alkyl group substituted with —F, a C₁-C₅ alkoxy group substituted with —F, and a C₂-C₅ alkenyl group substituted with —F; and
R₁₂ is selected from a hydrogen and a C₁-C₅ alkyl group.

2. The liquid crystal composition of claim 1, wherein 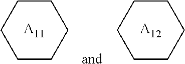
are each independently any one selected from Formulae 10-1 to 10-3 below: 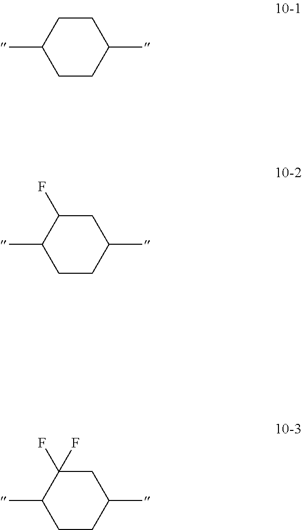
wherein in Formulae 10-1 to 10-3, ″ indicates a binding site to a neighboring atom.

3. The liquid crystal composition of claim 1, wherein 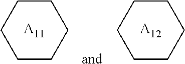
are each represented by Formula 10-1 below: 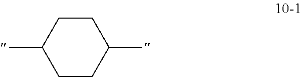
wherein in Formula 10-1, ″ indicates a binding site to a neighboring atom.

4. The liquid crystal composition of claim 1, wherein R₁₁ is selected from a hydrogen, a C₁-C₅ alkyl group, a C₁-C₅ alkoxy group, and a C₂-C₅ alkenyl group.

5. The liquid crystal composition of claim 1, wherein R₁₁ is selected from a methyl group, an ethyl group, a n-propyl group, a methoxy group, an ethoxy group, a n-propoxy group, an ethenyl group, and a 1-propenyl group.

6. The liquid crystal composition of claim 1, wherein R₁₂ is selected from a hydrogen, a methyl group, an ethyl group, a propyl group, a butyl group, and a pentyl group.

7. The liquid crystal composition of claim 1, wherein the first liquid crystal compound is represented by Formula 1-1 below: 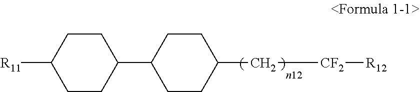
wherein in Formula 1-1, R₁₁, R₁₂, and n12 are the same as defined in connection with Formula 1.

8. The liquid crystal composition of claim 1, wherein the first liquid crystal compound is represented by any one of Compounds 10 to 34 below:

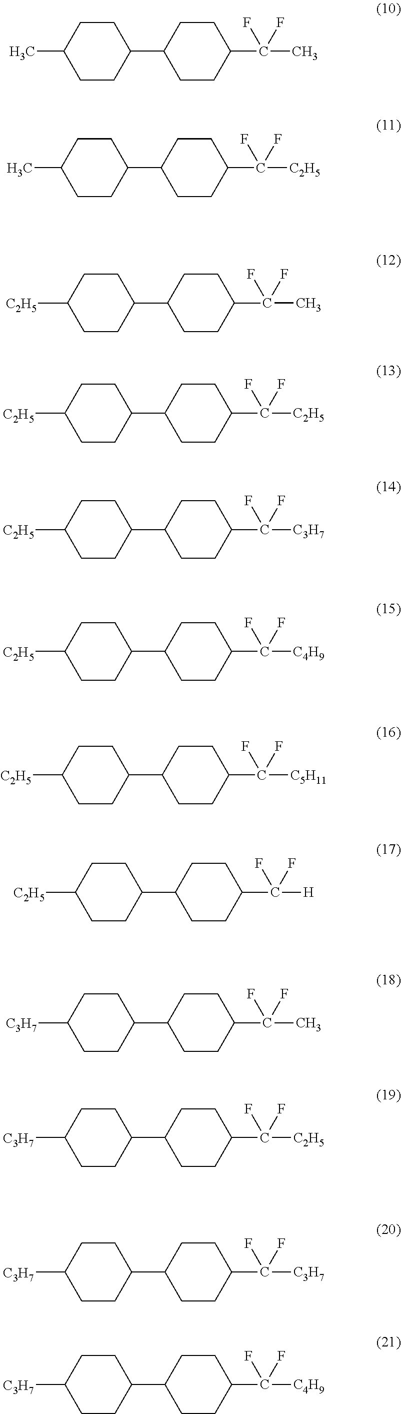

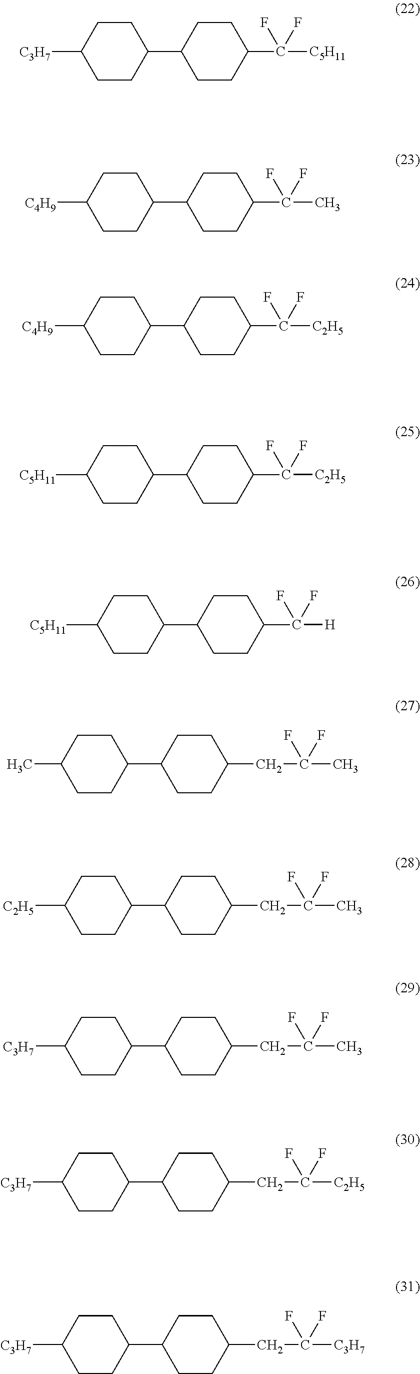

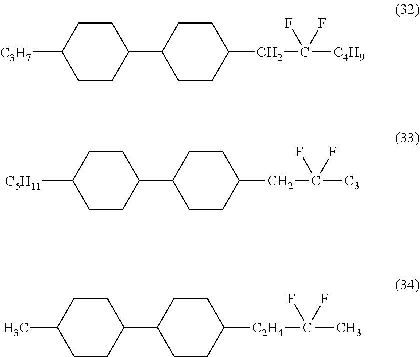

9. The liquid crystal composition of claim 1, wherein an amount of the first liquid crystal compound is in a range of about 0.1 to about 60 wt %.

10. The liquid crystal composition of claim 1, further comprising at least one of second liquid crystal compounds represented by Formula 2:
<Formula 2> 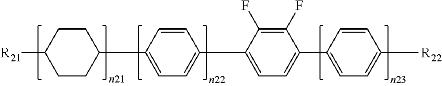
wherein in Formula 2, R₂₁ and R₂₂ are each independently selected from a C₁-C₇ alkyl group, a C₁-C₇ alkoxy group, and a C₂-C₇ alkenyl group;
n21, n22, and n23 are each independently selected from 0, 1, 2, and 3; and
the sum of n21, n22, and n23 is selected from 1, 2, and 3.

11. The liquid crystal composition of claim 10, wherein the second liquid crystal compound is represented by any one of Formulae 2-1 to 2-4 below: 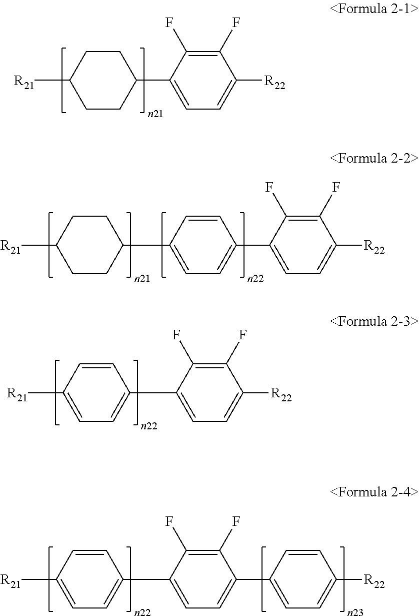
wherein in Formulae 2-1 to 2-4, R₂₁, R₂₂, n21, n22, and n23 are the same as defined in connection with Formula 2.

12. The liquid crystal composition of claim 10, wherein the second liquid crystal compound is represented by any one of Compounds 35 to 77 below:

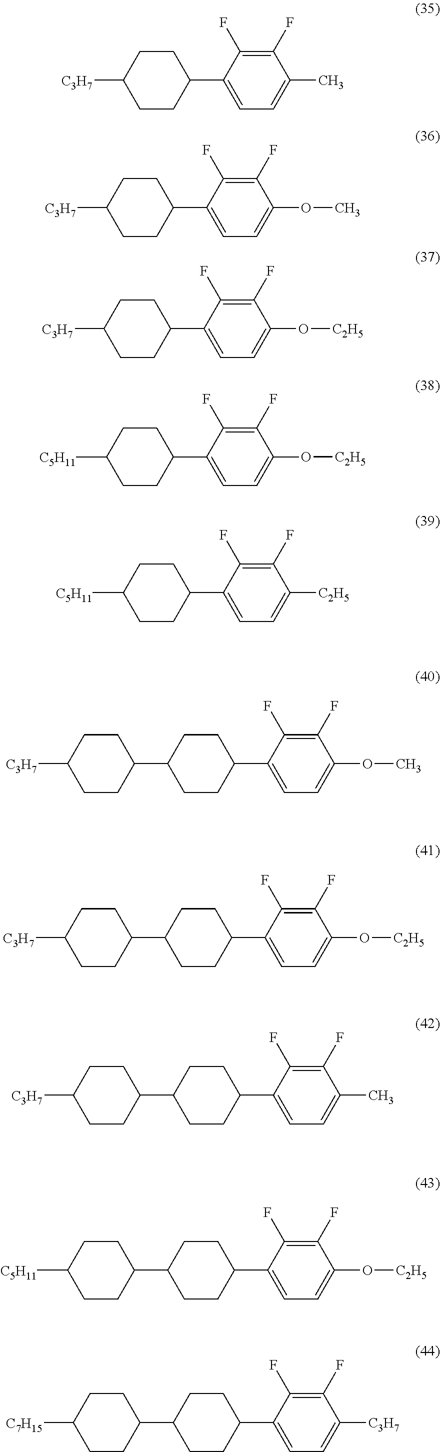

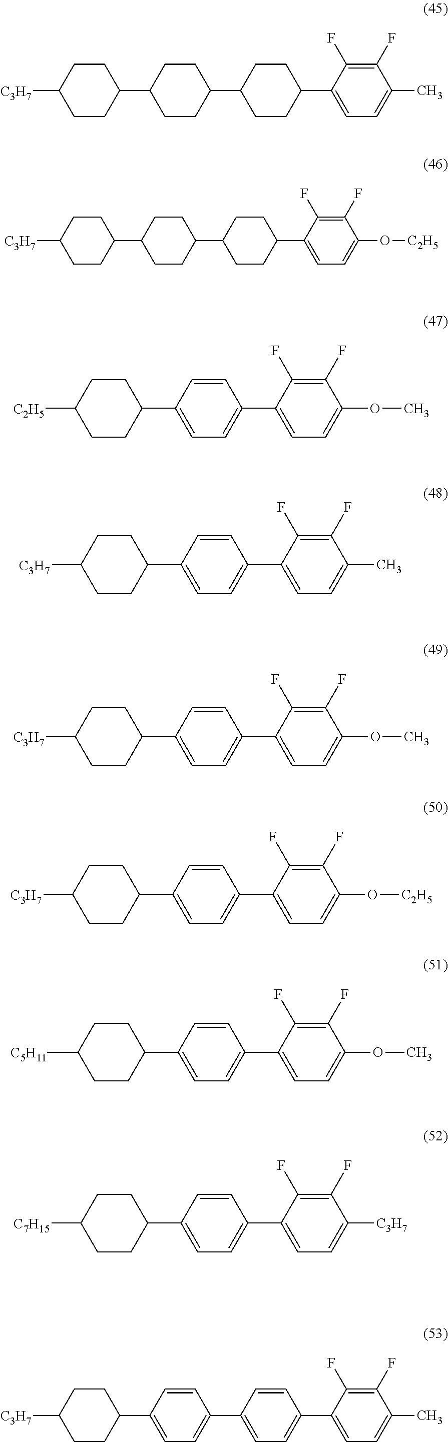

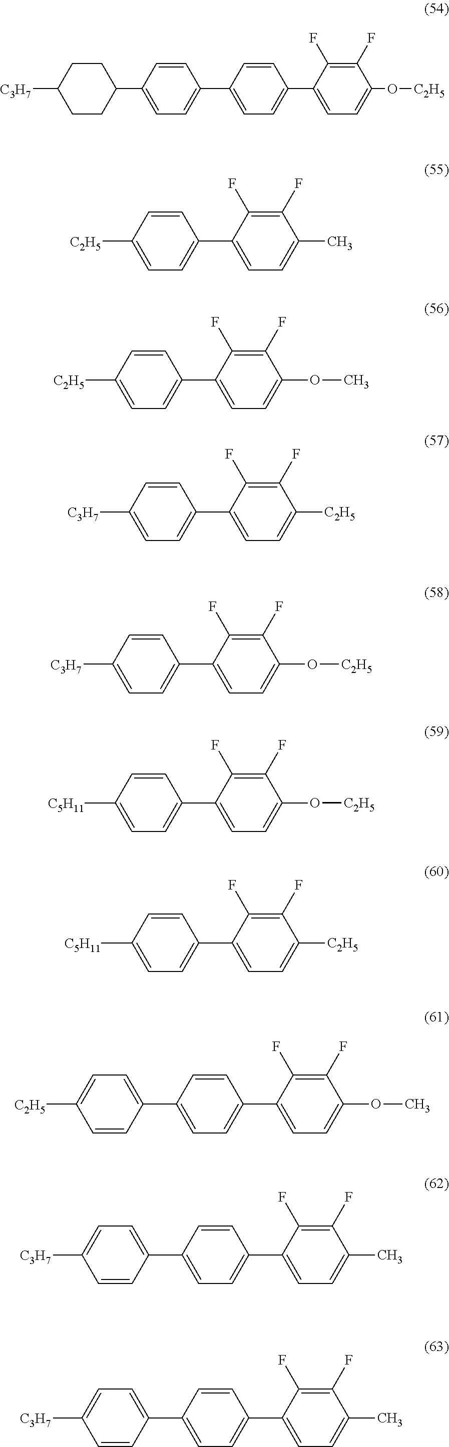

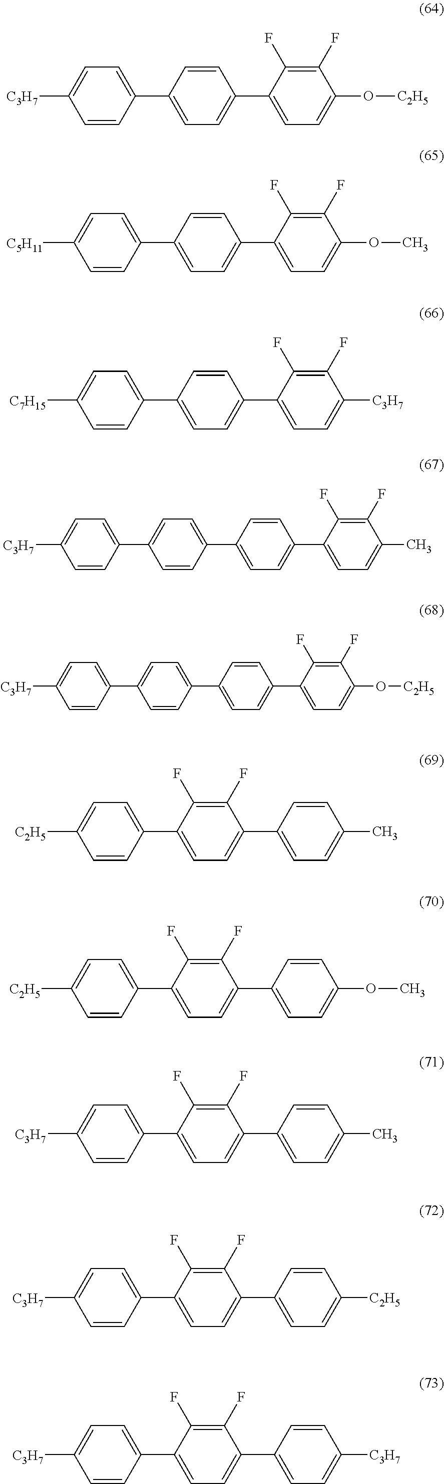

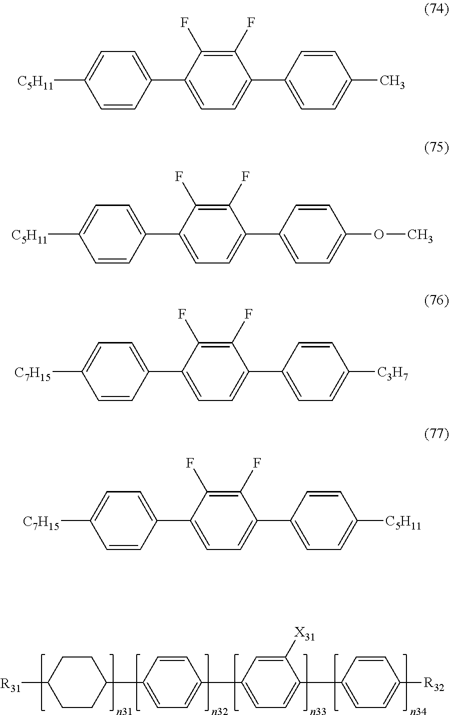

13. The liquid crystal composition of claim 1, further comprising at least one of third liquid crystal compounds represented by Formula 3:
<Formula 3>
wherein in Formula 3,
R₃₁ and R₃₂ are each independently selected from a C₁-C₇ alkyl group, a C₁-C₇ alkoxy group, and a C₂-C₇ alkenyl group;
X₃₁ is selected from a hydrogen and —F;
n31, n32, n33, and n34 are each independently selected from 0, 1, 2 and 3;
the sum of n31, n32, n33, and n34 is selected from 2, 3, and 4.

14. The liquid crystal composition of claim 13, wherein the third liquid crystal compound is represented by any one of Formulae 3-1 to 3-3 below: 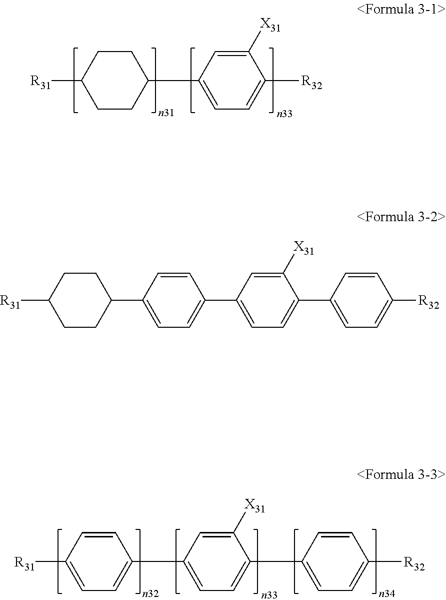
wherein in Formulae 3-1 to 3-3, R₃₁, R₃₂, X₃₁, n31, n32, n33, and n34 are the same as defined in connection with Formula 3.

15. The liquid crystal composition of claim 13, wherein the third liquid crystal compound is represented by any one of Compounds 78 to 119 below:

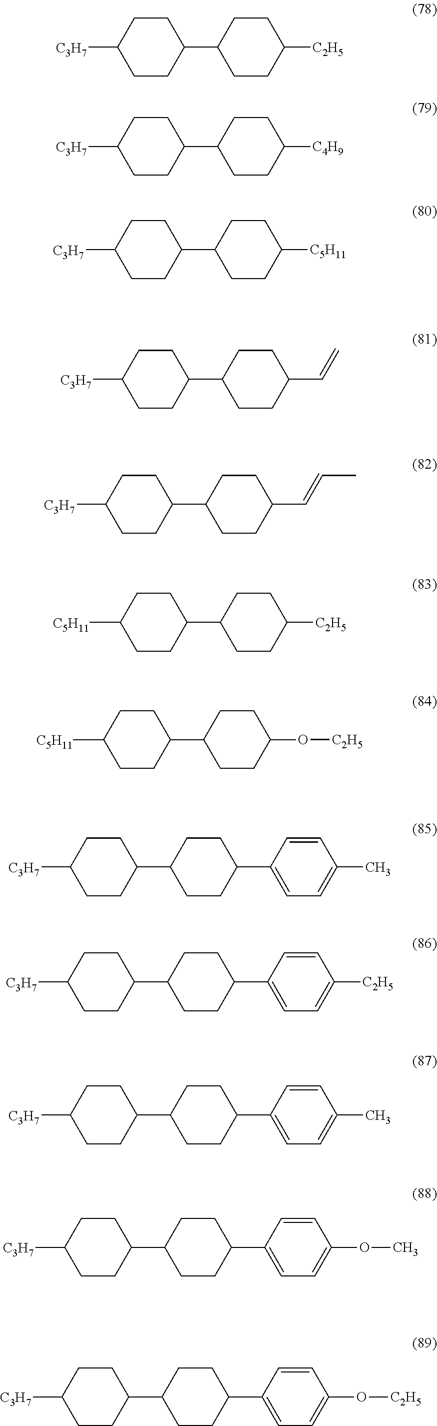

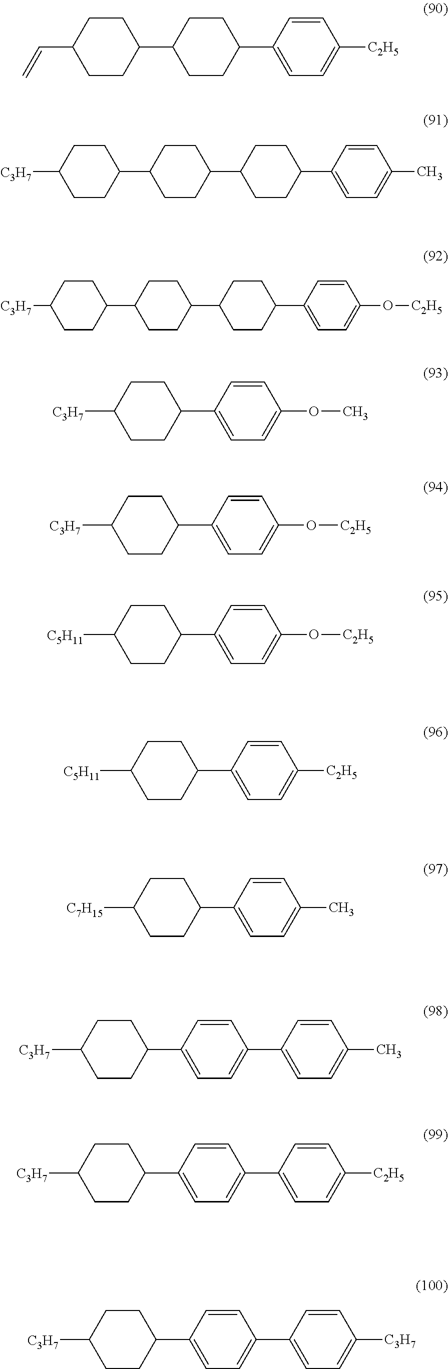

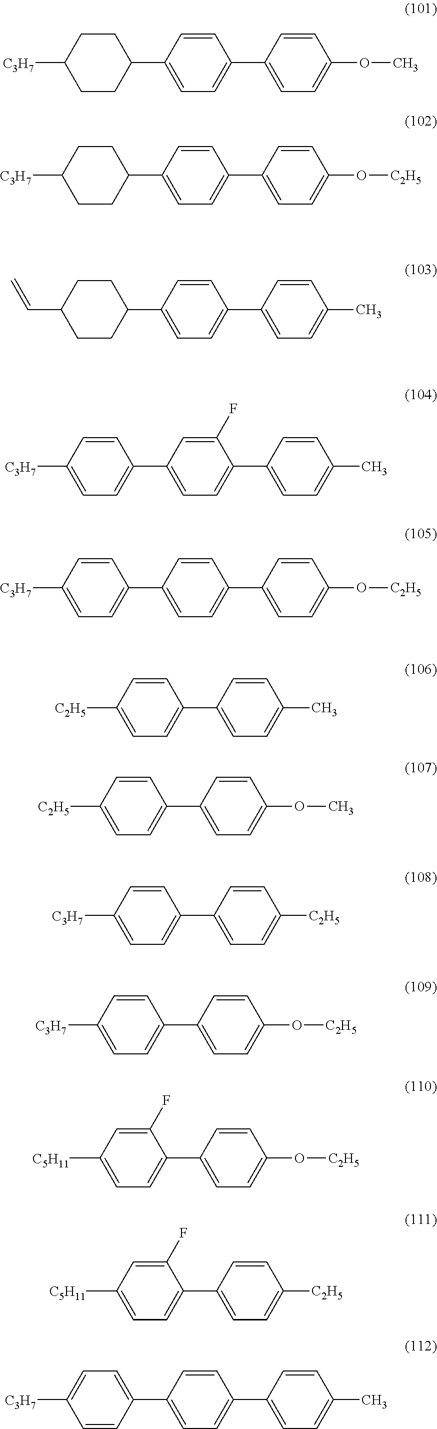

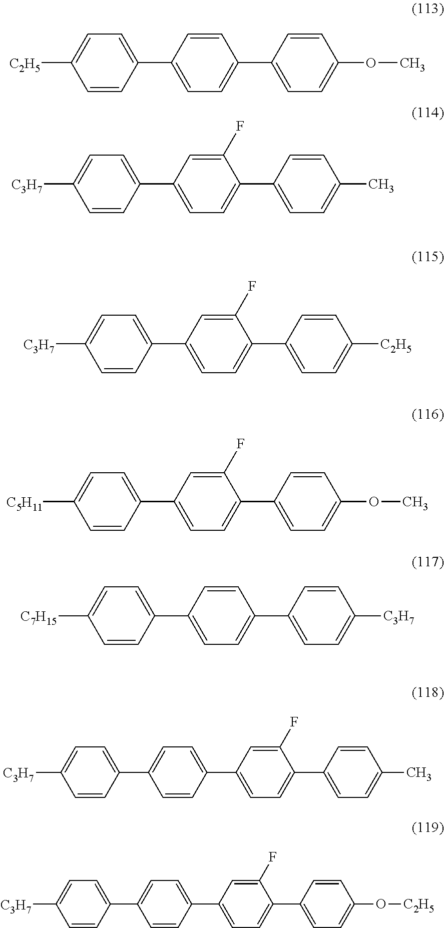

16. The liquid crystal composition of claim 1, further comprising at least one of fourth liquid crystal compounds represented by Formula 4 below: 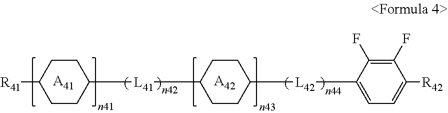
Wherein 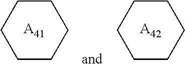
are each independently any one selected from Formulae 10-1 to 10-11 below; 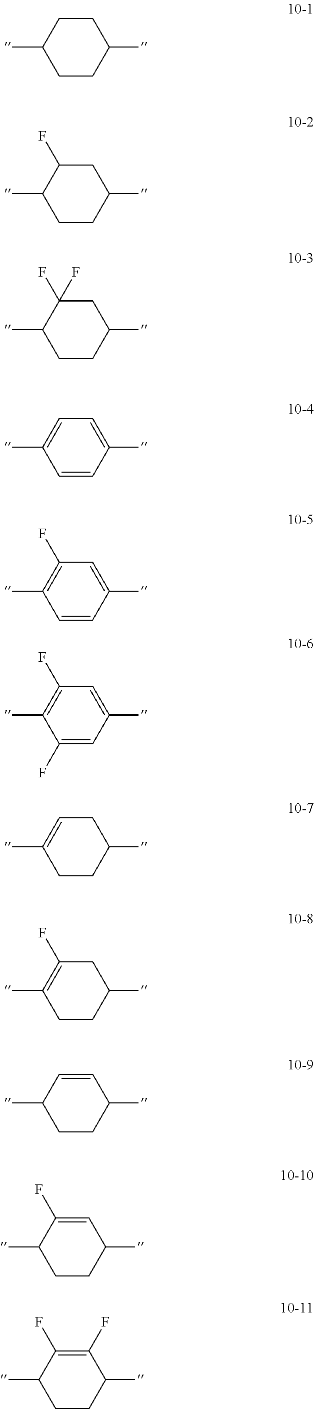
L₄₁ and L₄₂ are each independently selected from —CH₂CH₂—, —CH₂O—, —OCH₂—, —CF₂O—, and —OCF₂—;
R₄₁ and R₄₂ are each independently selected from a C₁-C₇ alkyl group, a C₁-C₇ alkoxy group, and a C₂-C₇ alkenyl group;
n41 is selected from 1, 2, and 3;
n42 to n44 are each independently selected from 0, 1, and 2;
the sum of n42 and n44 is selected from 1 and 2; and
″ in the formula above indicates a binding site to a neighboring atom.

17. The liquid crystal composition of claim 16, wherein the fourth liquid crystal compound is represented by Formula 4-1 below: 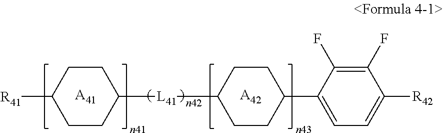
wherein in Formula 4-1, A₄₁, A₄₂, L₄₁, R₄₁, R₄₂, and n41 to n43 are the same as defined in connection with Formula 4.

18. The liquid crystal composition of claim 16, wherein the fourth liquid crystal compound is represented by any one of Compounds 120 to 134 below:

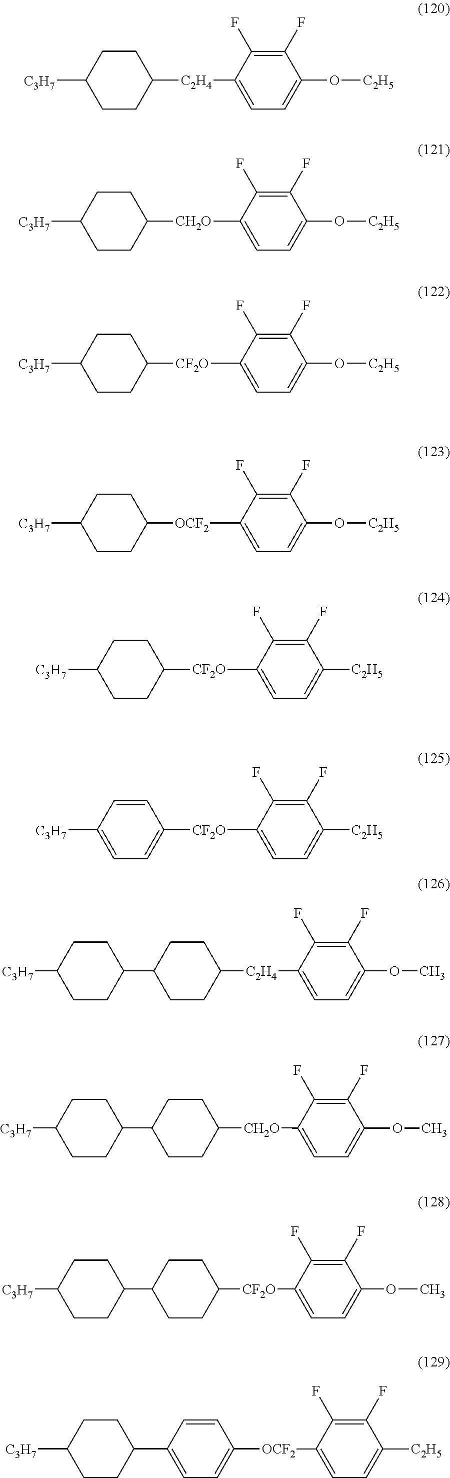

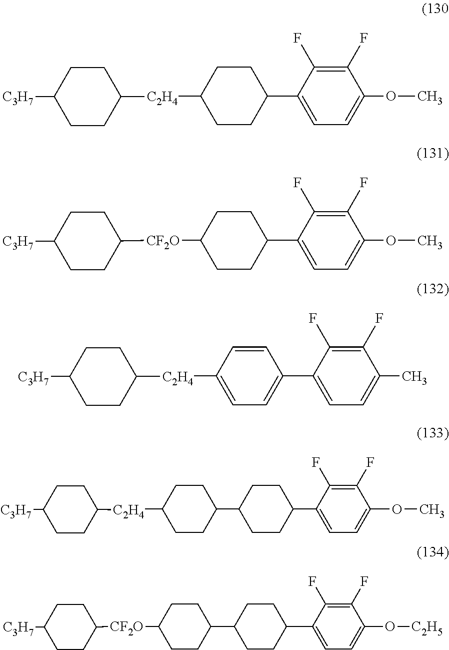

19. The liquid crystal composition of claim 1, further comprising at least one selected from a chiral agent, a polymerizable additive, and a stabilizing agent.

20. A liquid crystal display comprising:
a first electrode; a second electrode; and a liquid crystal layer between the first electrode and the second electrode, wherein
the liquid crystal layer comprises the liquid crystal composition of claim 1.
